(19) United States
(12) Patent Application Publication
(10) Pub. No.: US 2025/0197933 A1
(43) Pub. Date: Jun. 19, 2025

(54) HYDROGEL NANOPARTICLES AS LABELING SCAFFOLD IN SEQUENCING

(71) Applicant: ILLUMINA, INC., San Diego, CA (US)

(72) Inventors: Madushani Dharmarwardana, Cambridge (GB); Xiaolin Wu, Cambridge (GB); Xavier von Hatten, Cambridge (GB); Nam Trong Nguyen, Cambridge (GB)

(21) Appl. No.: 18/982,852

(22) Filed: Dec. 16, 2024

Related U.S. Application Data
(60) Provisional application No. 63/611,423, filed on Dec. 18, 2023.

(51) Int. Cl. C12Q 1/6874

(52) U.S. Cl. CPC C12Q 1/6874

(57) ABSTRACT

Embodiments of the present disclosure relate to compositions and methods for labeling of nucleotides, oligonucleotides, or polynucleotides with nanoparticles. In particular, labeled nanoparticles can be used to label nucleotides, oligonucleotides, or polynucleotides in sequencing by synthesis.

### CROSS-REFERENCE TO RELATED APPLICATION

The present application claims the benefit of priority to U.S. provisional application No. 63/611,423, filed Dec. 18, 2023, the content of which is incorporated by reference in its entirety.

### FIELD

The present disclosure generally relates to compositions, kits, methods and systems for nucleic acid sequencing applications.

### BACKGROUND

Nucleic acid sequencing methodology has evolved significantly from the chemical degradation methods used by Maxam and Gilbert and the strand elongation methods used by Sanger. Today several sequencing methodologies are in use which allow for the parallel processing of thousands of nucleic acids all in a single sequencing run. The instrumentation that performs such methods is typically large and expensive since the current methods typically rely on large amounts of expensive reagents and multiple sets of optic filters to record nucleic acid incorporation into sequencing reactions.

It has become clear that the need for high-throughput, smaller, less expensive DNA sequencing technologies will be beneficial for reaping the rewards of genome sequencing. Personalized healthcare is moving toward the forefront and will benefit from such technologies. The sequencing of an individual's genome to identify potential mutations and abnormalities will be crucial in identifying if a person has a particular disease, followed by subsequent therapies tailored to that individual. To accommodate such endeavor, sequencing technologies should not only have high throughput capabilities, but also have scalability. As such, there exists a need for new sequencing methods that improve on speed, error read, and are also cost effective.

### SUMMARY

The present disclosure provides next-generation sequencing kits, methods, systems, and compositions.

One aspect of the present disclosure relates to a labeled water-soluble nanoparticle, comprising a polymer matrix, the polymer matrix comprising one or more first functional moieties configured to bind to an unlabeled nucleotide, and a plurality of detectable labels, wherein the plurality of detectable labels are attached to the polymer matrix, optionally via covalent bonding to a plurality of second functional moieties of the polymer matrix or optionally via covalent bonding to the first functional moieties. In some embodiments, the polymer matrix comprises a hydrogel. In some embodiments, the first functional moiety is capable of forming covalent bonding with the unlabeled nucleotide via a biorthogonal reaction selected from the group consisting of a [3+2] dipolar cycloaddition, a Diels-Alder cycloaddition, a [4+1]cycloaddition, a phosphine ligation, or condensation with 2-acylphenyl boronic acid. In some other embodiments, the one or more first functional moieties are hapten-binding proteins capable of non-covalent binding with a hapten moiety of the unlabeled nucleotide. In some other embodiments, the one or more first functional moieties is a hapten moiety capable of non-covalent binding with hapten-binding proteins of the unlabeled nucleotide. In some further embodiments, the plurality of detectable labels are attached to the polymer matrix via covalent bonding to the plurality of second functional moieties of the polymer matrix.

Another aspect of the present disclosure relates to a method of determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an incorporation mixture comprising DNA polymerase and one more of four different types of nucleotides under conditions suitable for DNA polymerase-mediated primer extension, and incorporating one type of nucleotides into the sequencing primers to produce extended copy polynucleotides; wherein
each of the four types of nucleotides comprises a 3′ blocking group; and
the incorporation mixture comprises a first type of unlabeled nucleotide having a first binding moiety covalently attached to the first type of unlabeled nucleotide;
(c) contacting the extended copy polynucleotides with a first labeling reagent comprising the labeled water-soluble nanoparticle in accordance with the present disclosure that binds specifically with the first binding moiety of the first type of unlabeled nucleotides;
(d) imaging the solid support and performing one or more fluorescent measurements of the extended copy polynucleotides; and
(e) removing the 3′ blocking group of the incorporated nucleotides.

Another aspect of the present disclosure relates to a kit for sequencing application, comprising:
an incorporation mixture composition comprising one or more of four different types of nucleotides each comprising a 3′ blocking group, wherein a first type of unlabeled nucleotide having a first binding moiety covalently attached to the first type of unlabeled nucleotide; and
a first labeling reagent comprising a labeled water-soluble nanoparticle in accordance with the present disclosure that is capable of binding specifically with the first binding moiety of the first type of unlabeled nucleotides.

Another aspect of the present disclosure relates to a system for nucleic acid sequencing, comprising a plurality of chambers, wherein one of the chamber contains the kit in accordance with the present disclosure.

### BRIEF DESCRIPTION OF THE DRAWINGS

FIGS. 1A and 1B are cartoon illustrations of two exemplary nanoparticles in accordance with certain embodiments of the present disclosure. The nanoparticle of FIG. 1A includes detectable labels directly bound to the nanoparticle. The nanoparticle of FIG. 1B includes detectable labels bound to the functional moieties of the nanoparticle.

FIGS. 2A and 2B schematically illustrate steps of sequencing by synthesis in which an unlabeled nucleotide is incorporated into a growing polynucleotide chain and is subsequently labeled with a nanoparticle according to an embodiment of the present disclosure. The nanoparticle of FIG. 2A includes detectable labels directly bound to the nanoparticle. The nanoparticle of FIG. 2B includes detectable labels bound to the functional moieties of the nanoparticle.

FIGS. 3A and 3B are scatterplots of two-channel SBS runs conducted on MiSeq®. FIG. 3A was conducted with standard two-channel SBS reagents with pre-labeled ffNs. FIG. 3B was conducted with a set of ffNs with unlabeled fully functionalized C nucleotides (ffCs) having a biotin binding moiety and a nanoparticle with streptavidin functional groups that is capable of binding to the biotin moiety of the ffCs.

FIGS. 4A-4C are scatterplots of two-channel SBS runs conducted on MiSeq®. FIG. 4A was conducted with standard two-channel SBS reagents with pre-labeled ffNs. FIG. 4B was conducted with reagents including a set of ffNs with unlabeled ffCs having a biotin moiety and labeled streptavidin. FIG. 4C were conducted with reagents including a set of ffNs with unlabeled ffCs having a biotin moiety and streptavidin bound nanogel having detectable labels attached to streptavidin.

FIGS. 5A-5C are scatterplots of two-channel SBS runs conducted on MiSeq®. FIG. 5A was conducted with a set of ffNs including unlabeled ffC and a post-incorporation labeling reagent Tz-NR550C4. FIG. 5B and FIG. 5C were conducted with a set of ffNs including unlabeled ffCs and a labeled water-soluble nanoparticle Tz-PEG-17-NP-NR550C4 as a post-incorporation labeling reagent where the post-incorporation labeling incubation time was 50 s and 100 s respectively.

### DETAILED DESCRIPTION

### Definitions

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps but may include additional steps. When used in the context of a compound, composition, or device, the term “comprising” means that the compound, composition, or device includes at least the recited features or components, but may also include additional features or components.

As used herein, common organic abbreviations are defined as follows:
° C. Temperature in degrees Centigrade
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffA Fully functionalized A nucleotide
ffC Fully functionalized C nucleotide
ffG Fully functionalized G nucleotide
ffN Fully functionalized nucleotide
ffT Fully functionalized T nucleotide
LED Light emitting diode
SBS Sequencing by synthesis

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, or additionally, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,455,431 B1, US 2002/0102578 and PCT Publication No. WO 00/63437. Exemplary formats that can be used in the invention to distinguish beads in a liquid array, for example, using a microfluidic device, such as a fluorescent activated cell sorter (FACS), are described, for example, in U.S. Pat. No. 6,524,793. Further examples of arrays that can be used in the invention include, without limitation, those described in U.S. Pat. Nos. 5,429,807; 5,436,427; 5,561,071; 5,583,311; 5,658,734; 5,837,858; 5,874,319; 5,919,523; 6,236,369; 6,387,768; 6,387,776; 6,388,320; 6,397,006; 6,391,293; 6,446,413; 6,416,949; 6,482,591; 6,514,751 and 6,610,482; and WO 93/17126; WO 95/11995; WO 95/35505; EP 742 387; and EP 799 897.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, the term “non-covalent interactions” differs from a covalent bond in that it does not involve the sharing of electrons, but rather involves more dispersed variations of electromagnetic interactions between molecules or within a molecule. Non-covalent interactions can be generally classified into four categories, electrostatic, π-effects, van der Waals forces, and hydrophobic effects. Non-limiting examples of electrostatic interactions include ionic interactions, hydrogen bonding (a specific type of dipole-dipole interaction), halogen bonding, etc. Van der Walls forces are a subset of electrostatic interaction involving permanent or induced dipoles or multipoles. π-effects can be broken down into numerous categories, including (but not limited to) π-π interactions, cation-π & anion-π interactions, and polar-π interactions. In general, π-effects are associated with the interactions of molecules with the π-orbitals of a molecular system, such as benzene. The hydrophobic effect is the tendency of nonpolar substances to aggregate in aqueous solution and exclude water molecules. Non-covalent interactions can be both intermolecular and intramolecular. Non-covalent interactions can be both intermolecular and intramolecular.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Cₐ to C_{b},” “Cₐ-C_{b},” or “C_{a-b}” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b,” inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C₃, C₄, C₅ and C₆, and a range defined by any of the two numbers. For example, C₁-C₆ alkyl includes C₁, C₂, C₃, C₄, C₅ and C₆ alkyl, C₂-C₆ alkyl, C₁-C₃ alkyl, etc. Similarly, C₂-C₆ alkenyl includes C₂, C₃, C₄, C₅ and C₆ alkenyl, C₂-C₅ alkenyl, C₃-C₄ alkenyl, etc.; and C₂-C₆ alkynyl includes C₂, C₃, C₄, C₅ and C₆ alkynyl, C₂-C₅ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁-C₄ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁-C₉ alkoxy,” including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂-C₆ alkenyl” or similar designations. By way of example only, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁-C₆ alkylene group).

As used herein, “aryloxy” refers to RO— in which R is an aryl, as defined above, such as but not limited to phenyl.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinolinyl, benzoimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁-C₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “(aryl)alkyl” refer to an aryl group, as defined above, connected, as a substituent, via an alkylene group, as described above. The alkylene and aryl group of an aralkyl may be substituted or unsubstituted. Examples include but are not limited to benzyl, 2-phenylalkyl, 3-phenylalkyl, and naphthylalkyl. In some embodiments, the alkylene is an unsubstituted straight chain containing 1, 2, 3, 4, 5, or 6 methylene unit(s).

As used herein, “(heteroaryl)alkyl” refer to a heteroaryl group, as defined above, connected, as a substituent, via an alkylene group, as defined above. The alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, and imidazolylalkyl, and their benzo-fused analogs. In some embodiments, the alkylene is an unsubstituted straight chain containing 1, 2, 3, 4, 5, or 6 methylene unit(s).

As used herein, “(heterocyclyl)alkyl” refer to a heterocyclic or a heterocyclyl group, as defined above, connected, as a substituent, via an alkylene group, as defined above. The alkylene and heterocyclyl groups of a (heterocyclyl)alkyl may be substituted or unsubstituted. Examples include but are not limited to (tetrahydro-2H-pyran-4-yl)methyl, (piperidin-4-yl)ethyl, (piperidin-4-yl)propyl, (tetrahydro-2H-thiopyran-4-yl)methyl, and (1,3-thiazinan-4-yl)methyl. In some embodiments, the alkylene is an unsubstituted straight chain containing 1, 2, 3, 4, 5, or 6 methylene unit(s).

As used herein, “(carbocyclyl)alkyl” refer to a carbocyclyl group (as defined herein) connected, as a substituent, via an alkylene group. Examples include but are not limited to cyclopropylmethyl, cyclobutylmethyl, cyclopentylethyl, and cyclohexylpropyl. In some embodiments, the alkylene is an unsubstituted straight chain containing 1, 2, 3, 4, 5, or 6 methylene unit(s).

As used herein, “alkoxyalkyl” or “(alkoxy)alkyl” refers to an alkoxy group connected via an alkylene group, such as C₂-C₈ alkoxyalkyl, or (C₁-C₆ alkoxy)C₁-C₆ alkyl, for example, —(CH₂)₁₋₃—OCH₃.

As used herein, “—O-alkoxyalkyl” or “—O-(alkoxy)alkyl” refers to an alkoxy group connected via an —O-(alkylene) group, such as —O—(C₁-C₆ alkoxy)C₁-C₆ alkyl, for example, —O—(CH₂)₁₋₃—OCH₃.

As used herein, “haloalkyl” refers to an alkyl group in which one or more of the hydrogen atoms are replaced by ahalogen (e.g., mono-haloalkyl, di-haloalkyl, and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

An “amino” group refers to a —NH₂ group. The term “mono-substituted amino group” as used herein refers to an amino (—NH₂) group where one of the hydrogen atom is replaced by a substituent. The term “di-substituted amino group” as used herein refers to an amino (—NH₂) group where each of the two hydrogen atoms is replaced by a substituent. The term “optionally substituted amino,” as used herein refer to a —NR_{A}R_{B} group where R_{A} and R_{B} are independently hydrogen, alkyl, cycloalkyl, aryl, heteroaryl, heterocyclyl, aralkyl, or heterocyclyl(alkyl), as defined herein.

An “O-carboxy” group refers to a “—OC(═O)R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-carboxy” group refers to a “—C(═O)OR” group in which R is selected from the group consisting of hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes carboxyl (i.e., —C(═O)OH).

A “sulfonyl” group refers to an “—SO₂R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “sulfino” group refers to a “—S(═O)OH” group.

A “sulfo” group refers to a “—S(═O)₂OH” or “—SO₃H” group.

A “sulfonate” group refers to a “—SO₃” group.

A “sulfate” group refers to “—SO₄” group.

A “S-sulfonamido” group refers to a “—SO₂NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-sulfonamido” group refers to a “—N(R_{A})SO₂R_{B}” group in which R_{A} and Re are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “O-carbamyl” group refers to a “—OC(═O)N(R_{A}R_{B})” group in which R_{A} and R_{B} can be the same as defined with respect to S-sulfonamido. An O-carbamyl may be substituted or unsubstituted.

An “N-carbamyl” group refers to an “ROC(═O)N(R_{A})—” group in which R and R_{A} can be the same as defined with respect to N-sulfonamido. An N-carbamyl may be substituted or unsubstituted.

An “O-thiocarbamyl” group refers to a “—OC(═S)—N(R_{A}R_{B})” group in which R_{A} and R_{B} can be the same as defined with respect to S-sulfonamido. An O-thiocarbamyl may be substituted or unsubstituted.

An “N-thiocarbamyl” group refers to an “ROC(═S)N(R_{A})—” group in which R and R_{A} can be the same as defined with respect to N-sulfonamido. An N-thiocarbamyl may be substituted or unsubstituted.

The term “alkylamino” or “(alkyl)amino” refers to an amino group wherein one or both hydrogen is replaced by an alkyl group.

An “(alkoxy)alkyl” group refers to an alkoxy group connected via an alkylene group, such as a “(C₁-C₆ alkoxy) C₁-C₆ alkyl” and the like.

The term “hydroxy” as used herein refers to a “—OH” group.

The term “cyano” group as used herein refers to a “—CN” group.

The term “azido” as used herein refers to a “—N₃” group.

The term “isonitrile” as used herein refers to a “—N⁺≡C⁻” group.

When a group is described as “optionally substituted” it may be either unsubstituted or substituted. Likewise, when a group is described as being “substituted,” the substituent may be selected from one or more of the indicated substituents. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇ carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), (aryl)C₁-C₆ alkyl (optionally substituted with halo, C₁-C₆ alkyl, C₁-C₆ alkoxy, C₁-C₆haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), (5-10 membered heteroaryl)C₁-C₆ alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, (C₁-C₆ alkoxy)C₁-C₆ alkyl, —O(C₁-C₆ alkoxy)C₁-C₆ alkyl; (C₁-C₆ haloalkoxy)C₁-C₆ alkyl; —O(C₁-C₆ haloalkoxy)C₁-C₆ alkyl; aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, 0-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfonate, sulfate, sulfino, —OSO₂C₁₋₄alkyl, monophosphate, diphosphate, triphosphate, and oxo (═O). Wherever a group is described as “optionally substituted” that group can be substituted with the above substituents.

In each instance where a single mesomeric form of a compound described herein is shown, the alternative mesomeric forms are equally contemplated.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e., a sugar lacking a hydroxyl group that is present in ribose. The nitrogen containing heterocyclic base can be purine, deazapurine, or pyrimidine base. Purine bases include adenine (A) and guanine (G), and modified derivatives or analogs thereof, such as 7-deaza adenine or 7-deaza guanine. Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleotide conjugate” generally refers to a nucleotide labeled with a fluorescent moiety, optionally through a cleavage linker as described herein. In some embodiment, when a nucleotide conjugate is described as an unlabeled nucleotide, such nucleotide does not include a fluorescent moiety. In some further embodiments, an unlabeled nucleotide conjugate also does not have a cleavable linker.

As used herein, a “nucleoside” is structurally similar to a nucleotide but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art and includes its tautomers. Similarly, the term “pyrimidine base” is used herein in its ordinary sense as understood by those skilled in the art and includes its tautomers. A non-limiting list of optionally substituted purine-bases includes purine, adenine, guanine, deazapurine, 7-deaza adenine, 7-deaza guanine. hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, “derivative” or “analog” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, and phosphoramidate linkages. “Derivative,” “analog” and “modified” as used herein, may be used interchangeably, and are encompassed by the terms “nucleotide” and “nucleoside” defined herein.

As used herein, a dibenzocyclooxtyne (DBCO) moiety-containing molecule or an analog thereof comprises the structure

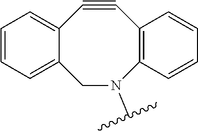 [SMILES: *N1Cc2ccccc2C#Cc2ccccc21]

such as

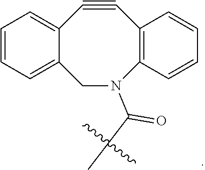 [SMILES: *C(=O)N1Cc2ccccc2C#Cc2ccccc21]

As used herein, the term “phosphate” is used in its ordinary sense as understood by those skilled in the art, and includes its protonated forms

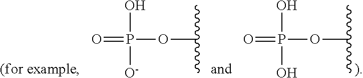 [SMILES: *OP(=O)([O-])O ; *OP(=O)(O)O]

As used herein, the terms “monophosphate,” “diphosphate,” and “triphosphate” are used in their ordinary sense as understood by those skilled in the art and include protonated forms.

As understood by one of ordinary skill in the art, a compound such as a nucleotide conjugate described herein may exist in ionized form, e.g., containing a —CO₂⁻, —SO₃⁻ or —O⁻. If a compound contains a positively or negatively charged substituent group, it may also contain a negatively or positively charged counterion such that the compound as a whole is neutral. In other aspects, the compound may exist in a salt form, where the counterion is provided by a conjugate acid or base.

As used herein, the term “phasing” refers to a phenomenon in SBS that is caused by incomplete removal of the 3′ terminators and fluorophores, and/or failure to complete the incorporation of a portion of DNA strands within clusters by polymerases at a given sequencing cycle. Prephasing is caused by the incorporation of nucleotides without effective 3′ terminators, wherein the incorporation event goes 1 cycle ahead due to a termination failure. Phasing and prephasing cause the measured signal intensities for a specific cycle to consist of the signal from the current cycle as well as noise from the preceding and following cycles. As the number of cycles increases, the fraction of sequences per cluster affected by phasing and prephasing increases, hampering the identification of the correct base. Prephasing can be caused by the presence of a trace amount of unprotected or unblocked 3′-OH nucleotides during sequencing by synthesis (SBS). The unprotected 3′-OH nucleotides could be generated during the manufacturing processes or possibly during the storage and reagent handling processes.

As used herein, the term “sequence context effect” or “sequence specific effect” refers to the effect that the intensity of a base as shown in a cloud scatterplot may be impacted by the preceding sequence context during sequencing by synthesis (SBS), in particular the two-channel SBS. This intensity modulation adds noise to the system and can cause miscalls when the sequence-specific intensity modulation shift a given cluster's intensity towards a decision boundary. It is also known as sequence specific errors (SSE). Without being bound by a particular theory, one reason for the sequence-specific intensity shifts is differential incorporation of fully functionalized nucleotides (ffNs) labeled with different dyes (e.g., green ffA and blue ffA). Another reason for causing fluorescent signal intensity shirt may be due to the preceding bases show different effect on the incorporated labeled nucleotides (e.g., quenching or enhancing dye signal intensity). As described in the present disclosure, using a single fully functionalized nucleotide (ffN) in connection with labeling reagent(s) that can produce color in two channels can reduce or eliminate sequence-specific intensity shifts and thereby improve base calling accuracy.

### Labeled Water-Soluble Nanoparticle

One aspect of the present disclosure relates to a labeled water-soluble nanoparticle. In one aspect, a labeled water-soluble nanoparticle can bind to a nucleotide, oligonucleotide, or polynucleotide. The labeled water-soluble nanoparticle can be used to label the nucleotide, oligonucleotide, or polynucleotide. The labeled water-soluble nanoparticle include:
a polymer matrix, the polymer matrix comprising:
one or more first functional moieties configured to bind to an unlabeled nucleotide, and
a plurality of detectable labels, wherein the plurality of detectable labels are attached to the polymer matrix, optionally via covalent bonding to a plurality of second functional moieties of the polymer matrix or optionally via covalent bonding to the first functional moieties.

In some embodiments, the polymer matrix comprises a hydrogel. In some embodiments, the first functional moiety is capable of forming covalent bonding with the unlabeled nucleotides via a biorthogonal reaction selected from the group consisting of a [3+2] dipolar cycloaddition, a Diels-Alder cycloaddition, a [4+1]cycloaddition, a phosphine ligation, or condensation with 2-acylphenyl boronic acid. In some further embodiments, one of the first functional moiety and the unlabeled nucleotide comprises alkynyl, unsubstituted or substituted dibenzocyclooctyne (DBCO) moiety, unsubstituted or substituted bicyclo[6.1.0]nonyne (BCN) moiety, unsubstituted or substituted norbornene moiety, unsubstituted or substituted transcyclooctene (TCO) moiety, primary isonitrile

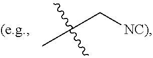

tertiary isonitrile

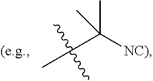

vinyl boronic acid

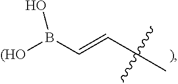

2-acylphenyl boronic acid

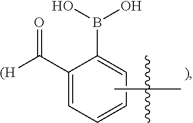

phosphine methyl ester

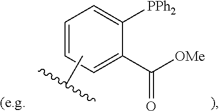

phosphine alcohol

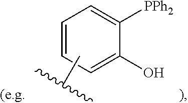

phosphine amine

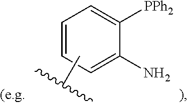

or phosphine thiol

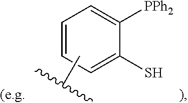

and the other one of the first functional moiety and the unlabeled nucleotide comprises azido, (e.g., primary azide such as

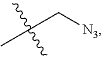 [SMILES: *CN=[N+]=[N-]]

aryl azide such as

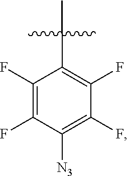 [SMILES: *c1c(F)c(F)c(N=[N+]=[N-])c(F)c1F]

tertiary azide such as

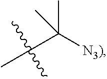

sydnone

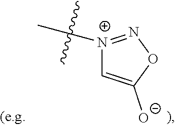 [SMILES: *[n+]1cc([O-])on1]

imino sydnone

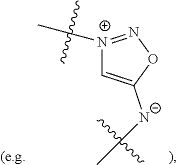 [SMILES: *[N-]c1c[n+](*)no1]

nitrone, phenyl tetrazine

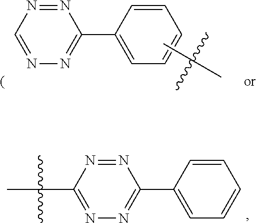

wherein the phenyl ring may be optionally substituted), pyrimidyl tetrazine

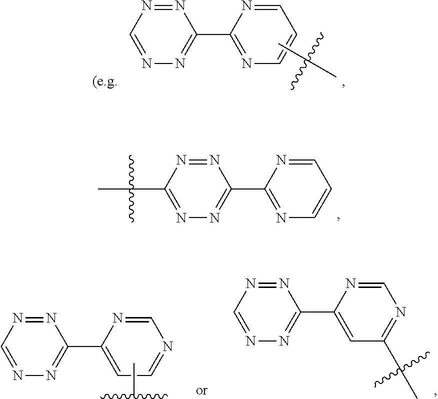

wherein the pyrimidyl ring may be optionally substituted), methyl tetrazine

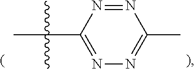 [SMILES: *c1nnc(C)nn1]

pyridyl tetrazine

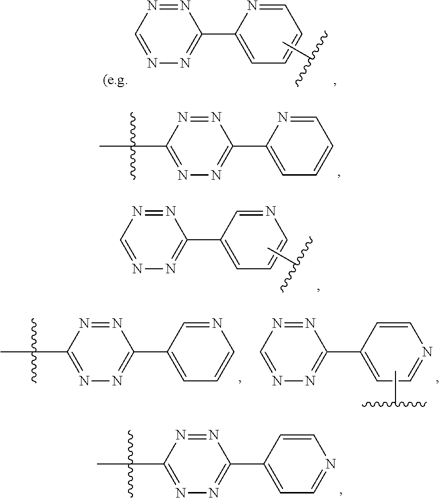

where the pyridyl ring may be optionally substituted), t-butyl tetrazine

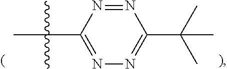 [SMILES: *c1nnc(C(C)(C)C)nn1]

triazine, cyclopropenone

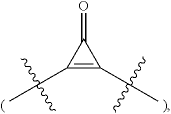 [SMILES: *c1c(*)c1=O]

cyclopropenium ion

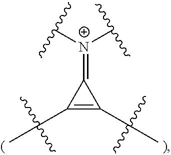 [SMILES: *c1c(*)c1=[N+](*)*]

### DTO

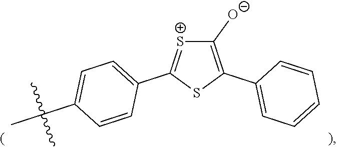 [SMILES: *c1ccc(-c2sc(-c3ccccc3)c([O-])[s+]2)cc1]

chloro-oxime

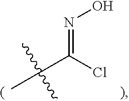 [SMILES: */C(Cl)=N/O]

or amino hydrazide

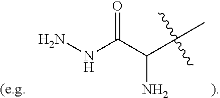 [SMILES: *C(N)C(=O)NN]

In some further embodiments, the one or more first functional moieties is covalently attached to the polymer matrix via a spacer. In yet further embodiments, the spacer comprises polyethylene glycol (PEG) units. In some further embodiments, the one or more first functional moieties comprises a 1,2,4,5-tetrazine moiety. In some embodiments, the 1,2,4,5-tetrazine moiety may be phenyl tetrazine

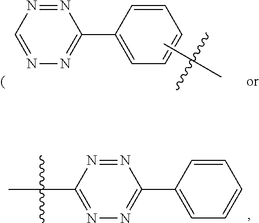

wherein the phenyl ring may be optionally substituted), pyrimidyl tetrazine (e.g.,

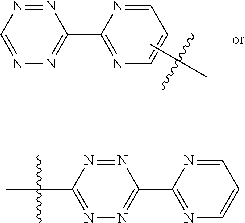

wherein the pyrimidyl ring is optionally substituted), methyl tetrazine

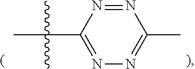 [SMILES: *c1nnc(C)nn1]

pyridyl tetrazine (e.g.,

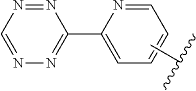

or

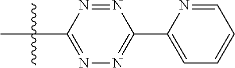 [SMILES: *c1nnc(-c2ccccn2)nn1]

where the pyridyl ring is optionally substituted), t-butyl tetrazine

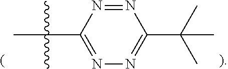 [SMILES: *c1nnc(C(C)(C)C)nn1]

In further embodiments, the one or more first functional moieties comprise a structure of

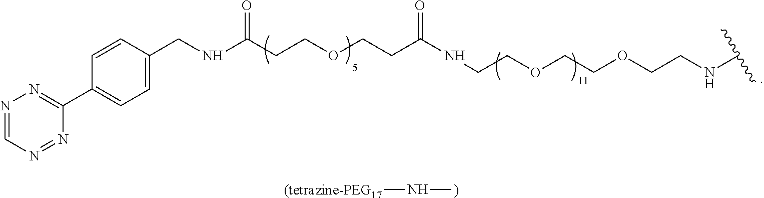

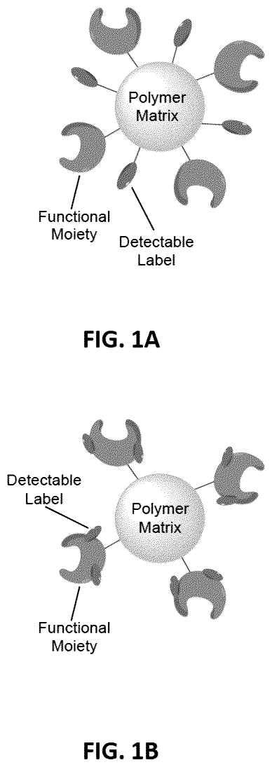

In some embodiments, the plurality of detectable labels are attached to the polymer matrix via covalent bonding to the plurality of second functional moieties of the polymer matrix. FIG. 1A is a cartoon illustration of an example nanoparticle where the detectable labels are attached to the polymer matrix via covalent bonding to the polymer matrix (i.e., direct bonding of the detectable label to the polymer matrix by reacting with secondary functional moieties of the polymer matrix). In further embodiments, the plurality of second functional moieties comprises carboxyl groups and/or amino groups.

In other embodiments, the one or more first functional moieties are hapten-binding proteins capable of non-covalent binding with a hapten moiety of the unlabeled nucleotide. In some further embodiments, the one or more first functional moieties comprise streptavidin or neutravidin. In yet further embodiments, the unlabeled nucleotide comprises a biotin moiety. In some embodiments, the plurality of detectable labels are attached to the polymer matrix via covalent bonding to the plurality of second functional moieties of the polymer matrix. In further embodiments, the plurality of second functional moieties comprises carboxyl groups and/or amino groups (e.g., the detectable label comprises amino and/or carboxyl groups such that carboxyl and amino groups can form an amide bond). In some embodiments, the plurality of detectable labels are attached to the polymer matrix via covalent bonding to the one or more first functional moieties. FIG. 1B is a cartoon illustration of an example nanoparticle where the detectable layers are attached to the polymer matrix via bonding to the first functional moieties. Such nanoparticles may be referred to herein as “indirect carriers.”

In some embodiments, the polymer matrix comprises a copolymer of polyacrylamide and polyacrylic acid. In further embodiments, the polymer matrix comprises a copolymerization product of N-isopropylacrylamide (NiPAM), acrylic acid (AAc) and N,N′-methylene bisacrylamide (BisAM). As such, the polymer matrix comprises carboxylic acid functional groups. In some such embodiments, the detectable label may comprise an amino group that can react with the carboxyl group of the polymer matrix.

In any of the embodiments described above, the plurality of detectable labels can include fluorophores excitable by a light source having a wavelength between about 450 nm to about 460 nm or about 520 nm to about 540 nm. In some embodiments, the detectable label comprises a structure of

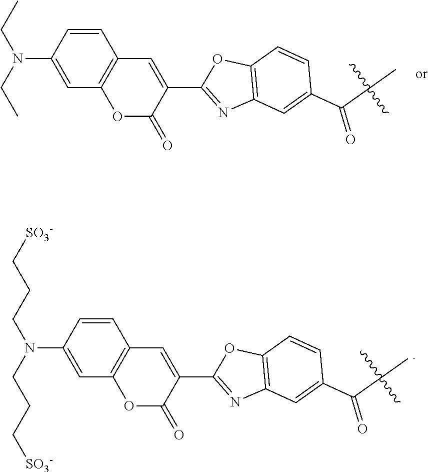

In some embodiments, the diameter of the labeled nanoparticle is about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, or 25 nm, or in a range defined by any two of the preceding values. In some embodiments, the diameter of the labeled nanoparticle is from 1 nm to 20 nm. In further embodiments, the diameter of the labeled nanoparticle is from 10 nm to 15 nm. In yet further embodiments, the diameter of the labeled nanoparticle is about 13 nm.

### Labeled Nucleotides

One aspect of the present disclosure relates to labeling of nucleotides, oligonucleotides, or polynucleotides using the labeled water-soluble nanoparticle. The one or more first functional moieties of the labeled water-soluble nanoparticle can bind to an unlabeled nucleotide, oligonucleotide, or polynucleotide. In some embodiments, the labeled water-soluble nanoparticle can bind to an unlabeled nucleotide, where the unlabeled nucleotide is a terminal nucleotide incorporated into an oligonucleotide or polynucleotide. In some further embodiments, the oligonucleotide or polynucleotide is hybridized to at least a portion of a target polynucleotide immobilized on a solid support. In yet further embodiments, the solid support comprises a plurality of different immobilized target polynucleotides.

In some embodiments, the labeled water-soluble nanoparticle is covalently bound to an unlabeled nucleotide, or a 3′ terminal unlabeled nucleotide moiety of the oligonucleotide or polynucleotide. In some further embodiments, the labeled water-soluble nanoparticle is covalently bound to the unlabeled nucleotide or the 3′ terminal unlabeled nucleotide moiety via a cleavable linker. In some embodiments, the oligonucleotide or polynucleotide is at least partially hybridized to a template polynucleotide immobilized on a solid support. In some embodiments, the solid support comprises an array of immobilized different template polynucleotides.

### 3′ Blocking Groups

The nucleotide described herein may also have a 3′ blocking group covalently attached to the deoxyribose sugar of the nucleotide. Various 3′ blocking group are disclosed in WO2002/029003, WO2004/018497 and WO2014/139596. For example, the blocking group may be azidomethyl (—CH₂N₃) or substituted azidomethyl (e.g., —CH(CHF₂)N₃ or CH(CH₂F)N₃), or allyl, each connecting to the 3′-oxygen atom of the deoxyribose moiety. In some embodiments, the 3′ blocking group is azidomethyl, forming 3′-OCH₂N₃ with the 3′ carbon of the ribose or deoxyribose.

Additional 3′ blocking groups are disclosed in U.S. Publication No. 2020/0216891 A1, which is incorporated by reference in its entirety. Non-limiting examples of the acetal blocking group

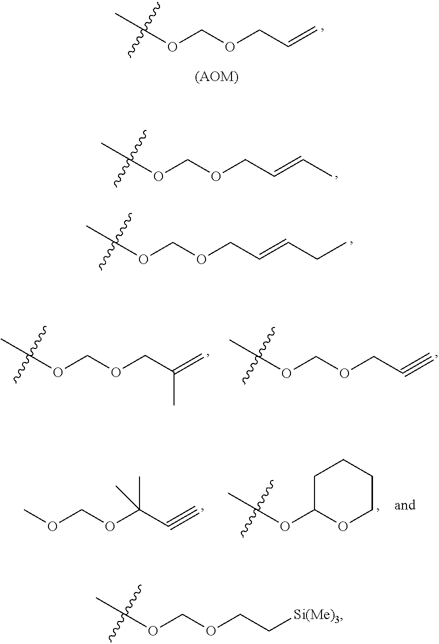

each covalently attached to the 3′ carbon of the deoxyribose.

### Deprotection of the 3′ Blocking Groups

In some embodiments, the 3′ hydroxy protecting group such as azidomethyl may be removed or deprotected by using a water-soluble phosphine reagent to generate a free 3′-OH. Non-limiting examples include tris(hydroxymethyl)phosphine (THMP), tris(hydroxyethyl)phosphine (THEP) or tris(hydroxypropyl)phosphine (THP or THPP). 3′-acetal blocking groups described herein may be removed or cleaved under various chemical conditions. For 3′ blocking groups that contain an allyl moiety, non-limiting cleaving condition includes a Pd(II) complex, such as Pd(OAc)₂ or allylPd(II) chloride dimer, in the presence of a phosphine ligand, for example tris(hydroxymethyl)phosphine (THMP), or tris(hydroxypropyl)phosphine (THP or THPP). For those blocking groups containing an alkynyl group (e.g., an ethynyl), they may also be removed by a Pd(II) complex (e.g., Pd(OAc)₂ or allyl Pd(II) chloride dimer) in the presence of a phosphine ligand (e.g., THP or THMP).

### Cleavable Linkers

As described herein, the reactive moiety or binding moiety of a nucleotide or a terminal unlabeled nucleotide moiety of the oligonucleotide or polynucleotide can be used to attach a nanoparticle. In some embodiments, the reactant moiety, configured to attach to the functional moiety of a nanoparticle, of nucleotides described herein is covalently attached to the nucleobase of the nucleotide via a cleavable linker. Use of the term “cleavable linker” is not meant to imply that the whole linker is required to be removed. The cleavage site can be located at a position on the linker that ensures that part of the linker remains attached to the nanoparticle and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

Particular linkers include those disclosed in PCT Publication No. WO2004/018493 (herein incorporated by reference) such as those that include moieties of the formulae:

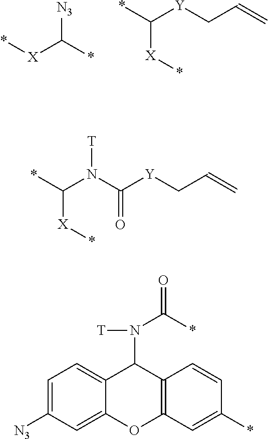

(wherein X is selected from the group comprising O, S, NH and NQ wherein Q is a C₁-C₁₀ substituted or unsubstituted alkyl group, Y is selected from the group comprising O, S, NH and N(allyl), T is hydrogen or a C₁-C₁₀ substituted or unsubstituted alkyl group and * indicates where the moiety is connected to the remainder of the nucleotide or nucleoside). In some aspect, the linkers connect the bases of nucleotides to labels such as, for example, the dye compounds described herein.

Additional examples of linkers include those disclosed in U.S. Publication No. 2016/0040225 (herein incorporated by reference), such as those include moieties of the formulae:

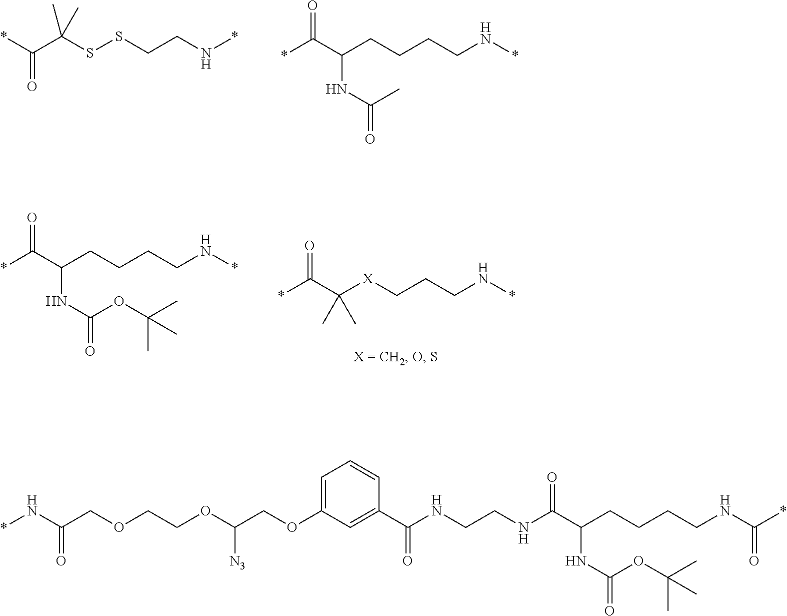

(wherein * indicates where the moiety is connected to the remainder of the nucleotide or nucleoside). The linker moieties illustrated herein may comprise the whole or partial linker structure between the nucleotides/nucleosides and the labels.

Additional examples of linkers are disclosed in U.S. Publication No. 2020/0216891 A1, which is incorporated by reference in its entirety:

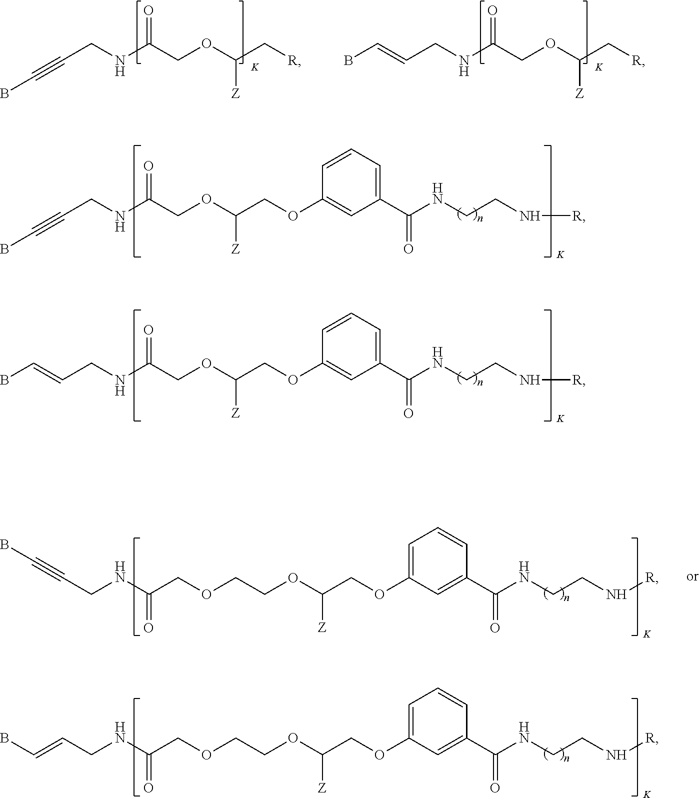

wherein B is a nucleobase; n is 1, 2, 3, 4, 5; k is 1; Z is —N₃ (azido), —O—C₁-C₆ alkyl, —O—C₂-C₆ alkenyl, or —O—C₂-C₆ alkynyl; and R comprises the first, the second, or the third binding moiety described herein, which may contain additional linker and/or spacer structure. One of ordinary skill in the art understands that the first, the second, or the third binding moiety described herein is covalently bound to the linker by reacting a functional group of the binding moiety containing compound (e.g., carboxyl) with a functional group of the linker (e.g., amino) to form an amide bond. In one embodiment, the cleavable linker comprises

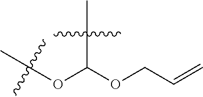 [SMILES: *OC(*)OCC=C]

(“AOL” linker moiety) where Z is —O-allyl. For the purpose of the present disclosure, the nucleotide may contain multiple cleavable linkers repeating units (e.g., k is 1, 2, 3, 4 5, 6, 7, 8, 9 or 10).

The first, second or third binding moiety may be attached to any position on the nucleotide base, for example, through a linker. In particular embodiments, Watson-Crick base pairing can still be carried out for the resulting analog. Particular nucleobase labeling sites include the C5 position of a pyrimidine base or the C7 position of a 7-deaza purine base. As described above a linker group may be used to covalently attach a dye to the nucleotide.

In particular embodiments, the unlabeled nucleotide may be enzymatically incorporable and enzymatically extendable. Accordingly, a linker moiety may be of sufficient length to connect the nucleotide to the compound such that the compound does not significantly interfere with the overall binding and recognition of the nucleotide by a nucleic acid replication enzyme. Thus, the linker can also comprise a spacer unit, such as one or more PEG unit(s)

(—OCH₂CH₂—)ₙ, where n is an integer of 1-20, for example 1, 2, 3, 4, 5, 6, 7, 8, 9, 10, 11, 12, 13, 14 or 15. The spacer distances, for example, the nucleotide base from a cleavage site or label.

A unlabeled nucleotides described herein may have the formula:

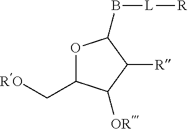

where R is the first, second or third binding moiety described herein; B is a nucleobase, such as, for example uracil, thymine, cytosine, adenine, 7-deaza adenine, guanine, 7-deaza guanine, and the like; L is a linker; —OR′ is monophosphate, diphosphate, triphosphate, thiophosphate, a phosphate ester analog, —O— attached to a reactive phosphorous containing group, or —O— protected by a blocking group; R″ is H or OH; and R′″ is H, a 3′ hydroxy blocking group described herein, or —OR′″ forms a phosphoramidite. Where —OR′″ is phosphoramidite, R′ is an acid-cleavable hydroxy protecting group which allows subsequent monomer coupling under automated synthesis conditions. In some further embodiments, B comprises

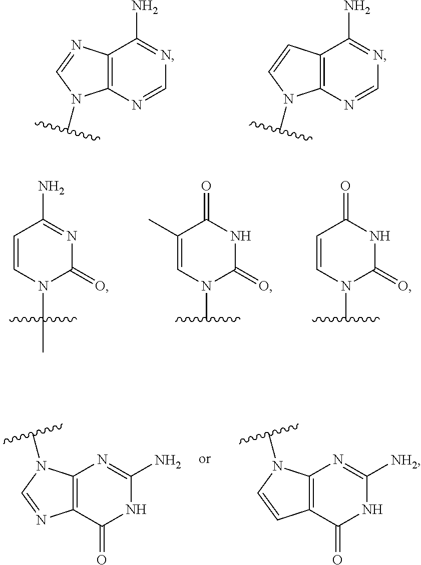

or optionally substituted derivatives and analogs thereof. In some further embodiments, the nucleobase comprises the structure

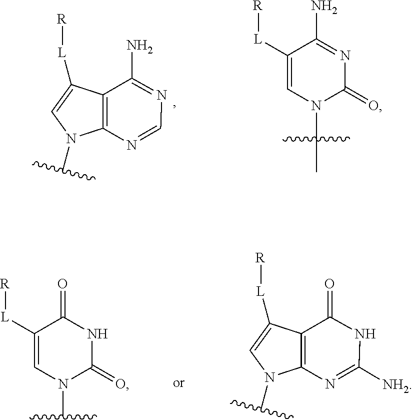

In yet another alternative embodiment, there is no blocking group on the 3′ carbon of the pentose sugar and the labeled avidin attached to the base via a linker, for example, can be of a size or structure sufficient to act as a block to the incorporation of a further nucleotide. Thus, the block can be due to steric hindrance or can be due to a combination of size, charge and structure, whether or not the dye is attached to the 3′ position of the sugar.

The use of a blocking group allows polymerization to be controlled, such as by stopping extension when an unlabeled nucleotide is incorporated. If the blocking effect is reversible, for example, by way of non-limiting example by changing chemical conditions or by removal of a chemical block, extension can be stopped at certain points and then allowed to continue.

In a particular embodiment, the linker and blocking group are both present and are separate moieties. In particular embodiments, the linker and blocking group are both cleavable under the same or substantially similar conditions. Thus, deprotection and deblocking processes may be more efficient because only a single treatment will be required to remove both the dye compound and the blocking group. However, in some embodiments a linker and blocking group need not be cleavable under similar conditions, instead being individually cleavable under distinct conditions.

Non-limiting exemplary unlabeled nucleotides as described herein include:

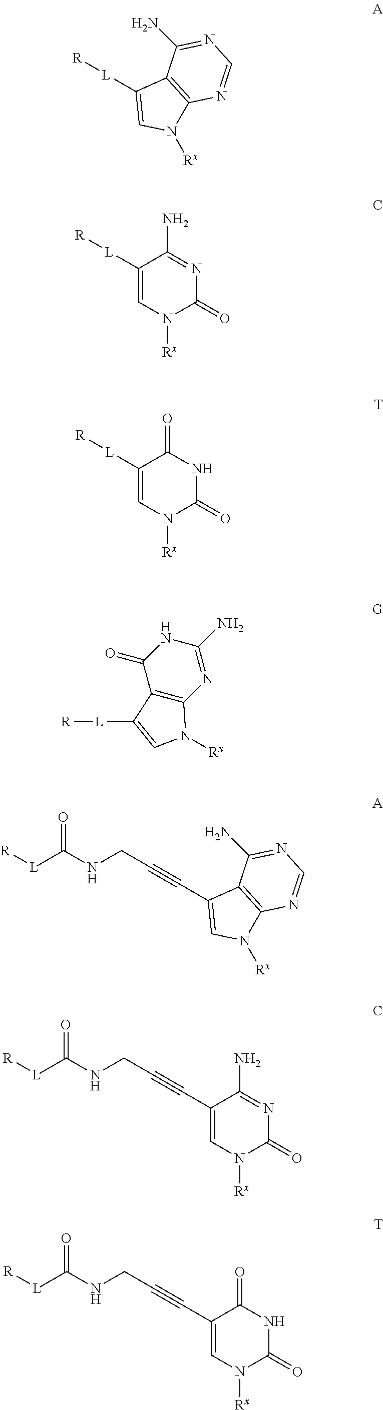

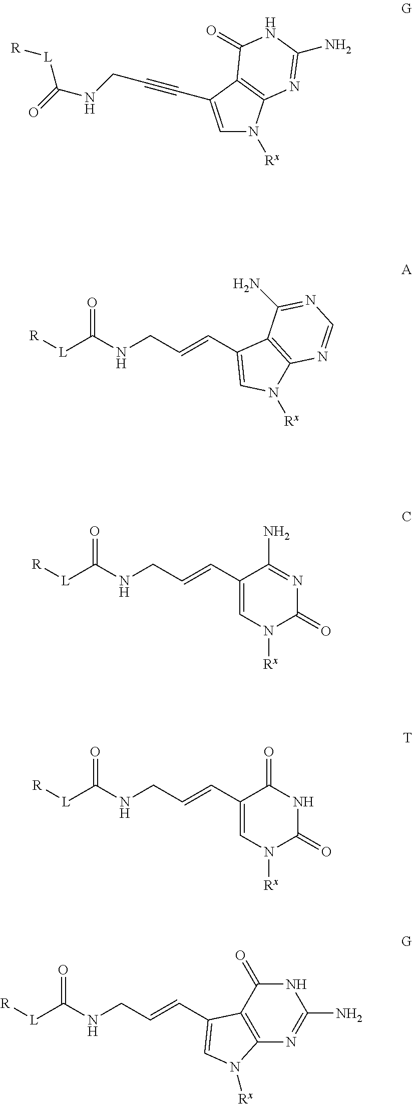

wherein L represents a linker, including a cleavable linker described herein; R^{x} represents a ribose or deoxyribose moiety as described above, or a ribose or deoxyribose moiety with the 5′ position substituted with mono-, di- or tri-phosphates; R represents the first, second or third binding moiety described herein.

In some embodiments, non-limiting exemplary unlabeled nucleotide containing a hapten moiety covalently attached via a cleavable linker are shown below:

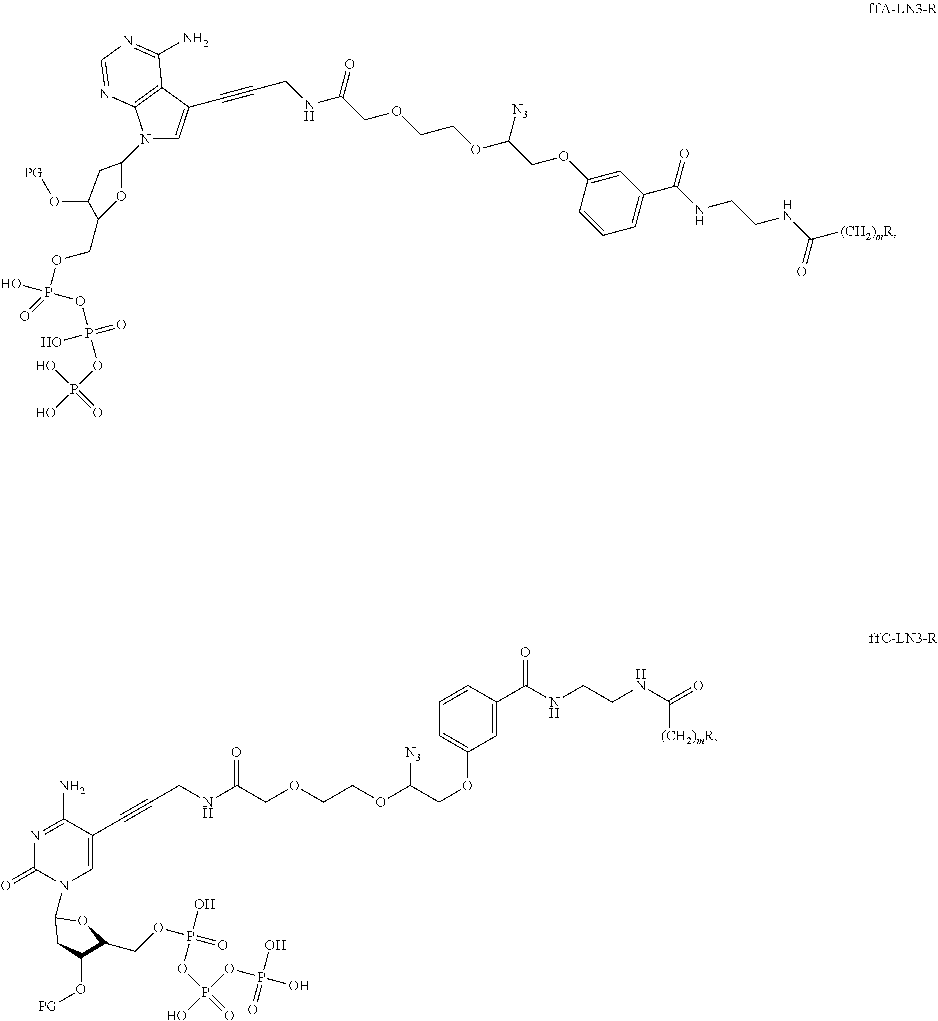

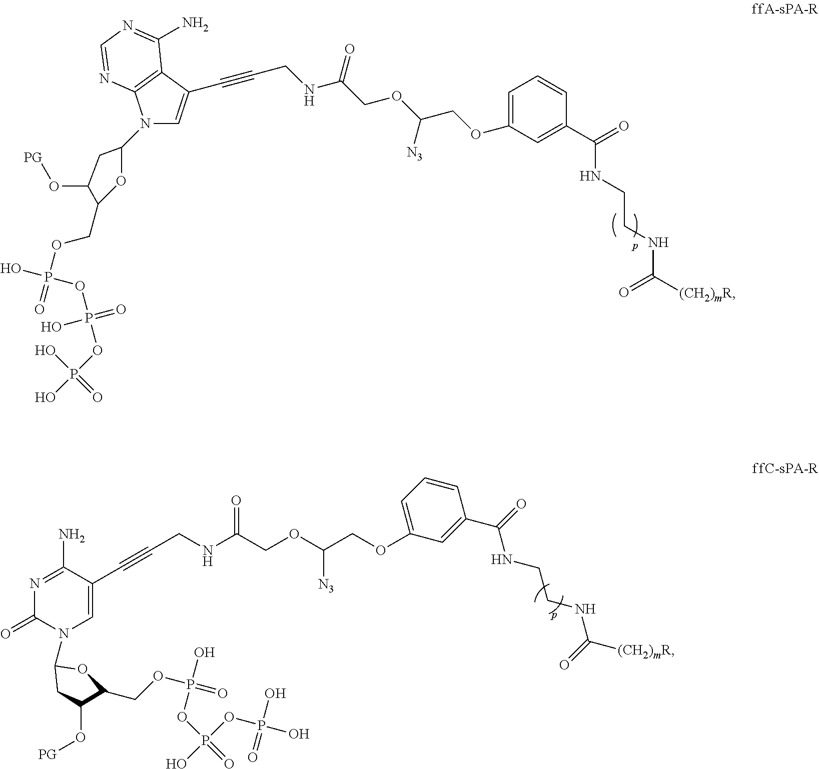

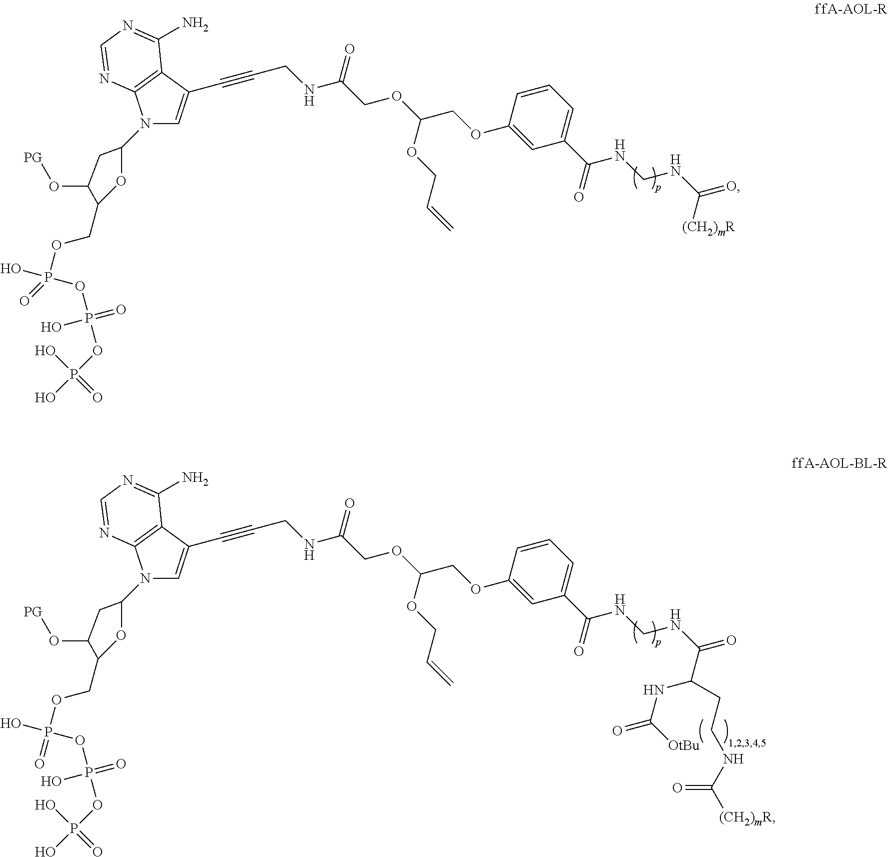

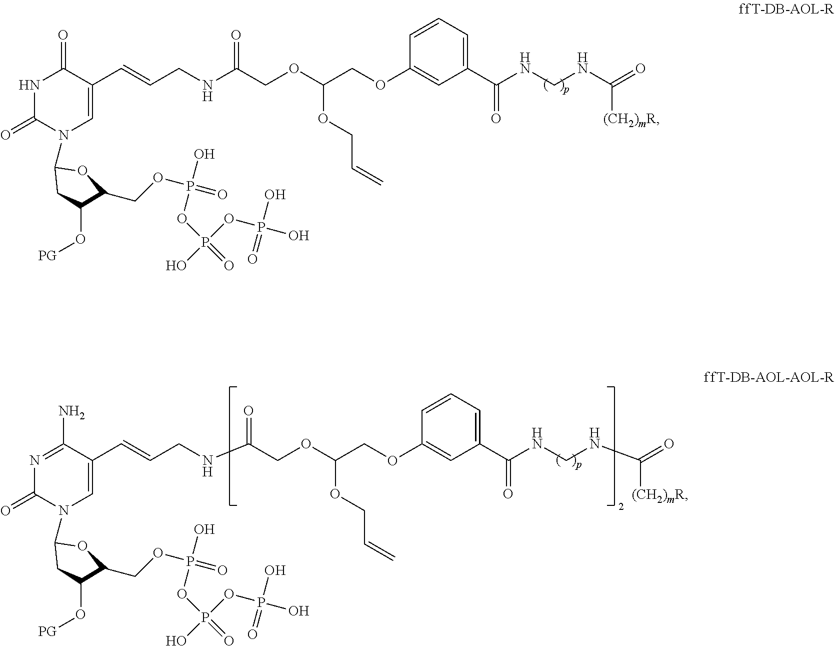

wherein PG stands for the 3′ blocking groups described herein; p is an integer of 1, 2, 3, 4, 5, 6, 7, 8, 9, or 10; and m is 0, 1, 2, 3, 4, or 5. In one embodiment, —O-PG is AOM. In another embodiment, —O-PG is —O-azidomethyl. In one embodiment, m is 5. In another embodiment, m is 0. In another embodiment, m is 2. In some further embodiments, p is 1, 2, 3, 4 or 5.

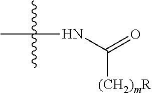

refers to the connection point of the first/second/third binding moiety with the cleavable linker as a result of a reaction between an amino group of the linker moiety and the carboxyl group of the first/second/third binding moiety. In further embodiments, the nucleotide may be attached to the binding moiety via more than one of the same cleavable linkers (such as LN3-LN3, sPA-sPA, AOL-AOL). In other embodiments, the nucleotide may be attached to the binding moiety via two or more different cleavable linkers (such sPA-LN3, sPA-sPA-LN3, sPA-LN3-LN3, etc.). In addition, the linker may further include additional PEG spacers as described herein, for example, between R and —(CH₂)ₘ—. In any embodiments of the labeled nucleotide described herein, the nucleotide is a nucleotide triphosphate. In further embodiments, the nucleotide has a 2′ deoxyribose.

### Methods of Sequencing

One aspect of the present disclosure relates to a method of determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an incorporation mixture comprising DNA polymerase and one more of four different types of nucleotides under conditions suitable for DNA polymerase-mediated primer extension, and incorporating one type of nucleotides into the sequencing primers to produce extended copy polynucleotides; wherein
each of the four types of nucleotides comprises a 3′ blocking group; and
the incorporation mixture comprises a first type of unlabeled nucleotide having a first binding moiety covalently attached to the first type of unlabeled nucleotide;
(c) contacting the extended copy polynucleotides with a first labeling reagent comprising the labeled water-soluble nanoparticle in accordance with the present disclosure that binds specifically with the first binding moiety of the first type of unlabeled nucleotides;
(d) imaging the solid support and performing one or more fluorescent measurements of the extended copy polynucleotides; and
(e) removing the 3′ blocking group of the incorporated nucleotides. In some embodiments, the first binding moiety is covalently attached to the nucleobase of the first type of unlabeled nucleotide via a cleavable linker.

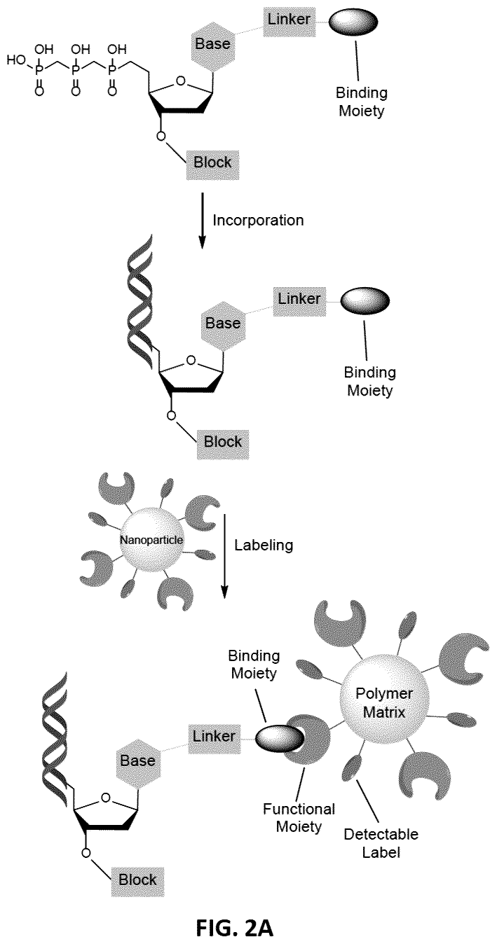

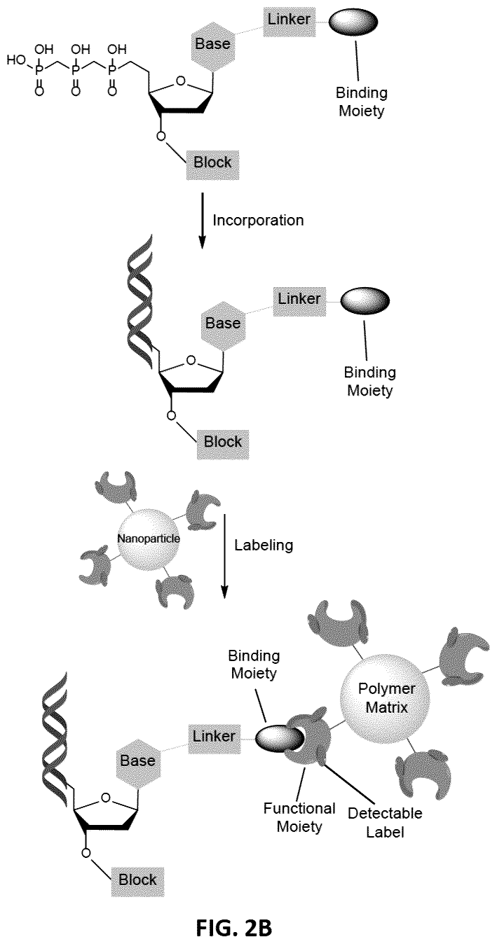

FIGS. 2A and 2B are cartoon drawings of an exemplary SBS schemes where a labeled water-soluble nanoparticle labels an incorporated nucleotide. A nucleotide having a binding moiety is incorporated into a DNA strand in the presence of a DNA polymerase. Then, a labeled water-soluble nanoparticle having a functional moiety is introduced, and the functional moiety binds to the binding moiety. In FIG. 2A, the nanoparticle has detectable labels which are directly bound to the polymer matrix. In FIG. 2B, the nanoparticle has detectable labels which are bound to the functional moiety (i.e., indirectly bound to the polymer matrix).

In some embodiments, the incorporation mixture comprises a second type of labeled nucleotide and a third type of labeled nucleotide. In other embodiments, the incorporation mixture comprises a second type of unlabeled nucleotide having a second binding moiety covalently attached to the second type of unlabeled nucleotide, and a third type of labeled nucleotide. In other embodiments, the incorporation mixture comprises a second type of unlabeled nucleotide having a second binding moiety covalently attached to the second type of unlabeled nucleotide, and a mixture of a third type of unlabeled nucleotide having a first binding moiety covalently attached to the third type of unlabeled nucleotide and a third type of unlabeled nucleotide having a second binding moiety covalently attached to the third type of unlabeled nucleotide. In some such embodiments, step (c) further comprises contacting the extended copy polynucleotides with a second labeling reagent comprising one or more second detectable labels and a functional moiety that binds specifically with the second binding moiety. In some embodiments, the first labeling reagent and the second labeling reagent are in a single aqueous solution (a labeling mixture). In other embodiments, the second labeling reagent may be introduced subsequent to the introduction of the first labeling reagent. In some embodiments, the incorporation mixture comprises a second type of unlabeled nucleotide having a second binding moiety covalently attached to the second type of unlabeled nucleotide, and a third type of unlabeled nucleotide having a third binding moiety covalently attached to the third type of unlabeled nucleotide. In some such embodiments, step (c) further comprises contacting the extended copy polynucleotides with a second labeling reagent comprising one or more second detectable labels and a moiety that binds specifically with a second functional moiety of the second type of unlabeled nucleotides, and a third labeling reagent comprising one or more third detectable labels and a functional moiety that binds specifically with a third binding moiety of the third type of unlabeled nucleotides. In some embodiments, the first labeling reagent, the second labeling reagent and the third labeling reagent are in a single aqueous solution (a labeling mixture). In other embodiments, the second and/or the third labeling reagent may be introduced subsequent to the introduction of the first labeling reagent.

In some embodiments, the incorporation mixture comprises a fourth type of unlabeled nucleotide, wherein the fourth type of unlabeled nucleotide is not capable of reacting with any of the labeling reagent. In some embodiments, step (e) also removes the detectable labels of the incorporated nucleotides. In further embodiments, the detectable labels and the 3′ blocking groups of the incorporated nucleotides are removed in a single chemical reaction. In some embodiments, the method further comprises (f) washing the solid support with an aqueous wash solution. In some embodiments, steps (b) to (f) are repeated at least 50, 100, 150, 200, 250, 300, 350, 400, 450, or 500 cycles to determine the target polynucleotide sequences. In some embodiments, the four types of nucleotides comprise dATP, dCTP, dGTP and dTTP or dUTP, or non-natural nucleotide analogs thereof. In some embodiments, the first labeling reagent is contacted with the extended copy polynucleotides in step (c) by flushing the first labeling reagent through the extended copy polynucleotides. In some embodiments, after step (b) the extended copy polynucleotides on the solid support are washed with a high salt buffer solution prior to contacting with the first labeling reagent. In some embodiments, after step (c) the extended copy polynucleotides on the solid support are washed with a buffer solution prior to imaging the solid support. In some embodiments, the method is performed on an automated sequencing instrument comprising two light sources operating at two different wavelengths. In further embodiments, one light source has a wavelength of about 450 nm to about 460 nm, and the other light source has a wavelength of about 520 nm to about 540 nm.

### Two-Tag Sequencing

In some embodiments where the third type of unlabeled nucleotide comprises a mixture of the third type of unlabeled nucleotide comprising the first binding moiety and the third type of unlabeled nucleotide comprising the second binding moiety, both the first labeling reagent and the second labeling reagent are capable of specific binding to the third type of unlabeled nucleotide. In some further embodiments, each of the first binding moiety and the second binding moiety is covalently attached to the nucleobase of the third type of nucleotide via a cleavable linker. In this instance, the incorporation of the first type of nucleotide is determined by a signal state in the first fluorescent measurement and a dark state in the second fluorescent measurement. The incorporation of the second type of nucleotide is determined by a dark state in the first fluorescent measurement and a signal state in the second fluorescent measurement. The incorporation of the third type of nucleotide is determined by a signal state in the first fluorescent measurement and a signal state in the second fluorescent measurement. The incorporation of the fourth type of nucleotide is determined by a dark state in the first fluorescent measurement and a dark state in the second fluorescent measurement.

In other embodiments, the incorporation mixture comprises a second type of labeled nucleotide and a third type of labeled nucleotide, in which the second type of nucleotide is labeled with a second detectable label that is spectrally distinguishable from the first detectable labels of the first labeling reagent. The third type of labeled nucleotide may be a mixture of the third type of nucleotide labeled with the first detectable label and the third type of nucleotide labeled with the second detectable label. In some such embodiments, the second detectable label is covalently attached to the nucleobase of the second type of nucleotide via a cleavable linker. In some such embodiments, the first and/or second detectable label is covalently attached to the nucleobase of the third type of nucleotide via a cleavable linker.

In other embodiments, the incorporation mixture comprises a second type of unlabeled nucleotide and a third type of labeled nucleotide, in which the second type of unlabeled nucleotide is labeled with a second labeling reagent post incorporation as described herein. The third type of labeled nucleotide may be a mixture of the third type of nucleotide labeled with the first detectable label and the third type of nucleotide labeled with the second detectable label. In some such embodiments, the first and/or second detectable label is covalently attached to the nucleobase of the third type of nucleotide via a cleavable linker.

The method described herein may also be referred to as a two-tag method as there are two labeling reagents (or two separate detectable labels), and the first detectable label (of the first labeling reagent) is spectrally distinguishable for the second detectable label (of the second labeling reagent). In some embodiments, the emissions of the first type of nucleotide, the second type of nucleotide and the third type of nucleotide are detectable in two detection channels with different wavelengths (e.g., at blue region with a wavelength ranging from about 472 to about 520 nm, and at a green region with a wavelength ranging from about 540 nm to about 640 nm).

### Three-Tag Sequencing

In some other embodiments, third type of unlabeled nucleotide comprises a third binding moiety, and the method further comprises a third labeling reagent comprising a third labeled water-soluble nanoparticle and one or more third detectable labels in accordance with the present disclosure. The third labeled water-soluble nanoparticle can be capable of specific binding to the third binding moiety of the third type of unlabeled nucleotide. In some such embodiments, the third binding moiety is covalently attached to the nucleobase of the third type of unlabeled nucleotide via a cleavable linker.

In other embodiments, the incorporation mixture comprises a second type of labeled nucleotide and a third type of labeled nucleotide, in which the second type of nucleotide is labeled with a second detectable label that is spectrally distinguishable from the first detectable labels of the first labeling reagent. The third type of labeled nucleotide contains a third detectable label that is spectrally distinguishable from both the first and the second detectable labels. In some such embodiments, the second detectable label is covalently attached to the nucleobase of the second type of nucleotide via a cleavable linker. In some such embodiments, the third detectable label is covalently attached to the nucleobase of the third type of nucleotide via a cleavable linker.

In other embodiments, the incorporation mixture comprises a second type of unlabeled nucleotide and a third type of labeled nucleotide, in which the second type of unlabeled nucleotide is labeled with a second labeling reagent post incorporation as described herein. The third type of labeled nucleotide contains a third detectable label that is spectrally distinguishable from both the first and the second detectable labels. In some such embodiments, the third detectable label is covalently attached to the nucleobase of the third type of nucleotide via a cleavable linker.

The sequencing system described herein may also be referred to as a three-tag system as there are three labeling reagents (or three separate detectable labels), each detectable label having spectrally distinguishable signature respectively.

In some embodiments, the sequencing method includes two imaging events, and the incorporation of the first type of nucleotide is determined by a signal state in the first fluorescent measurement and a dark state in the second fluorescent measurement. The incorporation of the second type of nucleotide is determined by a dark state in the first fluorescent measurement and a signal state in the second fluorescent measurement. The incorporation of the third type of nucleotide is determined by a signal state in the first fluorescent measurement and a signal state in the second fluorescent measurement. The incorporation of the fourth type of nucleotide is determined by a dark state in the first fluorescent measurement and a dark state in the second fluorescent measurement.

In some embodiments, the sequencing method includes a single imaging event. The incorporation of the first type of nucleotide is determined by a signal state in the first detection channel and a dark state in the second first detection channel. The incorporation of the second type of nucleotide is determined by a dark state in the first detection channel and a signal state in the second detection channel. The incorporation of the third type of nucleotide is determined by a signal state in the first detection channel and a signal state in the second detection channel. The incorporation of the fourth type of nucleotide is determined by a dark state in both the first and the second detection channels. Particular embodiments of the method of sequencing utilizes a one-excitation, two-channel detection system (also known as 1Ex-2Ch). Detailed disclosures are provided in WO 2018/165099 and U.S. 2022/0403450 A1, each of which is incorporated by reference in its entirety. In some embodiments, the 1Ex-2Ch may contain a blue excitation light source having a wavelength between about 450 nm to about 460 nm, and two separate collection channels at both the blue and green regions (e.g., at a blue region with a wavelength ranging from about 472 to about 520 nm, and at a green region with a wavelength ranging from about 540 nm to about 640 nm).

### The Fourth Type of Unlabeled Nucleotide

In any embodiments of the sequencing method described herein, the incorporation mixture further comprises a fourth type of unlabeled nucleotide, wherein the fourth type of unlabeled nucleotide is not capable of specific binding to any of the labeling reagents. Furthermore, the fourth type of unlabeled nucleotide cannot be excited by either the first or the second light source.

The term “signal state” when used in reference to an imaging event, refers to the state of a polynucleotide incorporating an unlabeled nucleotide and subsequently conjugate with a labeling reagent described herein, in which a specific emission signal is produced by such imaging event, and the emission signal is measured, detected or collected. For example, one or more detectable labels of the labeling reagent specifically bound to the incorporated unlabeled nucleotide may be excited by a light source (e.g., a laser) at a specific wavelength and emits a fluorescent signal that is collected or detected in the single emission detection channel/filter, indicating a “signal state” in such imaging event and fluorescent measurement.

The term “dark state,” when used in reference to an imaging event, refers to the state of a polynucleotide incorporating an unlabeled nucleotide, and subsequently conjugate with a labeling reagent described herein, in which no specific emission signal is produced by such imaging event, or no emission signal is measured, collected, and/or detected.

In one example of the method described herein, “C” nucleotide conjugate is determined by a signal state in the first fluorescent measurement and a dark state in the second fluorescent measurement; “T” nucleotide conjugate is determined by a dark state in the first fluorescent measurement and a signal state in the second fluorescent measurement; “A” nucleotide conjugate is determined by a signal state in the first fluorescent measurement and a signal state in the second fluorescent measurement; and “G” nucleotide conjugate is determined by a dark state in the first fluorescent measurement and a dark state in the second fluorescent measurement. In another example, “T” nucleotide conjugate is determined by a signal state in the first fluorescent measurement and a dark state in the second fluorescent measurement; “C” nucleotide conjugate is determined by a dark state in the first fluorescent measurement and a signal state in the second fluorescent measurement; “A” nucleotide conjugate is determined by a signal state in the first fluorescent measurement and a signal state in the second fluorescent measurement; and “G” nucleotide conjugate is determined by a dark state in the first fluorescent measurement and a dark state in the second fluorescent measurement.

### General Description on Sequencing by Synthesis

In a specific embodiment, a synthetic step is carried out and may optionally comprise incubating a template or target polynucleotide strand with a reaction mixture comprising fluorescently labeled nucleotides of the disclosure. A polymerase can also be provided under conditions which permit formation of a phosphodiester linkage between a free 3′ hydroxy group on a polynucleotide strand annealed to the template or target polynucleotide strand and a 5′ phosphate group on the labeled nucleotide. Thus, a synthetic step can include formation of a polynucleotide strand as directed by complementary base pairing of nucleotides to a template/target strand.

In all embodiments of the methods, the detection step may be carried out while the polynucleotide strand into which the labeled nucleotides are incorporated is annealed to a template/target strand, or after a denaturation step in which the two strands are separated. Further steps, for example chemical or enzymatic reaction steps or purification steps, may be included between the synthetic step and the detection step. In particular, the polynucleotide strand incorporating the labeled nucleotide(s) may be isolated or purified and then processed further or used in a subsequent analysis. By way of example, the polynucleotide strand incorporating the labeled nucleotide(s) as described herein in a synthetic step may be subsequently used as labeled probes or primers. In other embodiments, the product of the synthetic step set forth herein may be subject to further reaction steps and, if desired, the product of these subsequent steps purified or isolated.

Suitable conditions for the synthetic step will be well known to those familiar with standard molecular biology techniques. In one embodiment, a synthetic step may be analogous to a standard primer extension reaction using nucleotide precursors, including the labeled nucleotides as described herein, to form an extended polynucleotide strand (primer polynucleotide strand) complementary to the template/target strand in the presence of a suitable polymerase enzyme. In other embodiments, the synthetic step may itself form part of an amplification reaction producing a labeled double stranded amplification product comprised of annealed complementary strands derived from copying of the primer and template polynucleotide strands. Other exemplary synthetic steps include nick translation, strand displacement polymerization, random primed DNA labeling, etc. A particularly useful polymerase enzyme for a synthetic step is one that is capable of catalyzing the incorporation of the labeled nucleotides as set forth herein. A variety of naturally occurring or mutant/modified polymerases can be used. By way of example, a thermostable polymerase can be used for a synthetic reaction that is carried out using thermocycling conditions, whereas a thermostable polymerase may not be desired for isothermal primer extension reactions. Suitable thermostable polymerases which are capable of incorporating the labeled nucleotides according to the disclosure include those described in WO 2005/024010 or WO06120433, each of which is incorporated herein by reference. In synthetic reactions which are carried out at lower temperatures such as 37° C., polymerase enzymes need not necessarily be thermostable polymerases, therefore the choice of polymerase will depend on a number of factors such as reaction temperature, pH, strand-displacing activity and the like.

In specific non-limiting embodiments, the disclosure encompasses methods of nucleic acid sequencing, re-sequencing, whole genome sequencing, single nucleotide polymorphism scoring, any other application involving the detection of the modified nucleotide or nucleoside labeled with dyes set forth herein when incorporated into a polynucleotide.

SBS generally involves sequential addition of one or more nucleotides or oligonucleotides to a growing polynucleotide chain in the 5′ to 3′ direction using a polymerase or ligase in order to form an extended polynucleotide chain complementary to the template/target nucleic acid to be sequenced. The identity of the base present in one or more of the added nucleotide(s) can be determined in a detection or “imaging” step. The identity of the added base may be determined after each nucleotide incorporation step. The sequence of the template may then be inferred using conventional Watson-Crick base-pairing rules. The use of the nucleotides labeled with dyes set forth herein for determination of the identity of a single base may be useful, for example, in the scoring of single nucleotide polymorphisms, and such single base extension reactions are within the scope of this disclosure.

In an embodiment of the present disclosure, the sequence of a template/target polynucleotide is determined by detecting the incorporation of one or more nucleotides into a nascent strand complementary to the template polynucleotide to be sequenced through the detection of fluorescent label(s) attached to the incorporated nucleotide(s). Sequencing of the template polynucleotide can be primed with a suitable primer (or prepared as a hairpin construct which will contain the primer as part of the hairpin), and the nascent chain is extended in a stepwise manner by addition of nucleotides to the 3′ end of the primer in a polymerase-catalyzed reaction.

In particular embodiments, each of the different nucleotide triphosphates (A, T, G and C) may be labeled with a unique fluorophore and also comprises a blocking group at the 3′ position to prevent uncontrolled polymerization. Alternatively, one of the four nucleotides may be unlabeled (dark). The polymerase enzyme incorporates a nucleotide into the nascent chain complementary to the template/target polynucleotide, and the blocking group prevents further incorporation of nucleotides. Any unincorporated nucleotides can be washed away and the fluorescent signal from each incorporated nucleotide can be “read” optically by suitable means, such as a charge-coupled device using light source excitation and suitable emission filters. The 3′-blocking group and fluorescent dye compounds can then be removed (deprotected) (simultaneously or sequentially) to expose the nascent chain for further nucleotide incorporation.

The method, as exemplified above, utilizes the incorporation of fluorescently labeled, 3′ blocked nucleotides A, G, C, and T into a growing strand complementary to the immobilized polynucleotide, in the presence of DNA polymerase. The polymerase incorporates a base complementary to the target polynucleotide but is prevented from further addition by the 3′ blocking group. The label of the incorporated nucleotide can then be determined, and the blocking group removed by chemical cleavage to allow further polymerization to occur. The nucleic acid template to be sequenced in a SBS reaction may be any polynucleotide that it is desired to sequence. The nucleic acid template for a sequencing reaction will typically comprise a double stranded region having a free 3′ hydroxy group that serves as a primer or initiation point for the addition of further nucleotides in the sequencing reaction. The region of the template to be sequenced will overhang this free 3′ hydroxy group on the complementary strand. The overhanging region of the template to be sequenced may be single stranded but can be double-stranded, provided that a “nick” is present on the strand complementary to the template strand to be sequenced to provide a free 3′ OH group for initiation of the sequencing reaction. In such embodiments, sequencing may proceed by strand displacement. In certain embodiments, a primer bearing the free 3′ hydroxy group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Publication Nos. WO0157248 and WO2005/047301, each of which is incorporated herein by reference. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′ hydroxy group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

In certain embodiments, the nucleic acid template to be sequenced may be attached to a solid support via any suitable linkage method known in the art, for example via covalent attachment. In certain embodiments template polynucleotides may be attached directly to a solid support (e.g., a silica-based support). However, in other embodiments of the disclosure the surface of the solid support may be modified in some way so as to allow either direct covalent attachment of template polynucleotides, or to immobilize the template polynucleotides through a hydrogel or polyelectrolyte multilayer, which may itself be non-covalently attached to the solid support.

Arrays in which polynucleotides have been directly attached to a support (for example, silica-based supports such as those disclosed in WO00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO2005/047301 (incorporated herein by reference). A still further example of solid-supported template polynucleotides is where the template polynucleotides are attached to hydrogel supported upon silica-based or other solid supports, for example, as described in WO00/31148, WO01/01143, WO02/12566, WO03/014392, U.S. Pat. No. 6,465,178 and WO00/53812, each of which is incorporated herein by reference.

A particular surface to which template polynucleotides may be immobilized is a polyacrylamide hydrogel. Polyacrylamide hydrogels are described in the references cited above and in WO2005/065814, which is incorporated herein by reference. Specific hydrogels that may be used include those described in WO2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Template(s) that are to be sequenced may form part of an “array” on a solid support, in which case the array may take any convenient form. Thus, the method of the disclosure is applicable to all types of high-density arrays, including single-molecule arrays, clustered arrays, and bead arrays. Nucleotides labeled with dye compounds of the present disclosure may be used for sequencing templates on essentially any type of array, including but not limited to those formed by immobilization of nucleic acid molecules on a solid support.

However, nucleotides labeled with dye compounds of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using nucleotides labeled with dye compounds of the disclosure.

Nucleotides labeled with dye compounds of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO00/06770 and WO 01/57248, each of which is incorporated herein by reference. Although one use of the labeled nucleotides of the disclosure is in SBS reactions, the utility of such nucleotides is not limited to such methods. In fact, the labeled nucleotides described herein may be used advantageously in any sequencing methodology which requires detection of fluorescent labels attached to nucleotides incorporated into a polynucleotide.

In particular, nucleotides labeled with dye compounds of the disclosure may be used in automated fluorescent sequencing protocols, particularly fluorescent dye-terminator cycle sequencing based on the chain termination sequencing method of Sanger and co-workers. Such methods generally use enzymes and cycle sequencing to incorporate fluorescently labeled dideoxynucleotides in a primer extension sequencing reaction. So-called Sanger sequencing methods, and related protocols (Sanger-type), utilize randomized chain termination with labeled dideoxynucleotides.

### Kits

One aspect of the present disclosure relates to a kit for sequencing application, comprising:
an incorporation mixture composition comprising one or more of four different types of nucleotides each comprising a 3′ blocking group, wherein a first type of unlabeled nucleotide having a first binding moiety covalently attached to the first type of unlabeled nucleotide; and
a first labeling reagent comprising a labeled water-soluble nanoparticle according to the present disclosure that is capable of binding specifically with the first binding moiety of the first type of unlabeled nucleotides. In some embodiments, the first binding moiety is covalently attached to the nucleobase of the first type of unlabeled nucleotide via a cleavable linker.

In some embodiments, the kit comprises a second type of labeled nucleotide, and a third type of labeled nucleotide. In other embodiments, the kit comprises a second type of unlabeled nucleotide having a second binding moiety covalently attached to the second type of unlabeled nucleotide, and a third type of labeled nucleotide. In yet other embodiments, the kit comprises a second type of unlabeled nucleotide having a second binding moiety covalently attached to the second type of unlabeled nucleotide, and a mixture of a third type of unlabeled nucleotide having a first binding moiety covalently attached to the third type of unlabeled nucleotide and a third type of unlabeled nucleotide having a second binding moiety covalently attached to the third type of unlabeled nucleotide. In some embodiments, the kit further comprises a second labeling reagent comprising one or more second detectable labels and a functional moiety that is capable of reacting specifically with the second binding moiety to form covalent bonding. In some embodiments, the kit comprises a second type of unlabeled nucleotide having a second binding moiety covalently attached to the second type of unlabeled nucleotide, and a third type of unlabeled nucleotide having a third binding moiety covalently attached to the third type of unlabeled nucleotide. In further embodiments, the kit further comprises a second labeling reagent comprising one or more second detectable labels and a functional moiety that is capable of reacting specifically with the second binding moiety of the second type of unlabeled nucleotide, and a third labeling reagent comprising one or more third detectable labels and a functional moiety that is capable of reacting specifically with the third binding moiety of the third type of unlabeled nucleotide. In some embodiments, the kit comprises a fourth type of unlabeled nucleotide, wherein the fourth type of unlabeled nucleotide is not capable of reacting with any labeling reagent. In some embodiments, the incorporation mixture composition further comprises a DNA polymerase (such as a mutant 9° N DNA polymerase) and/or one or more buffer compositions. Non-limiting examples of DNA polymerase may be used in the present disclosure include those disclosed in WO 2005/024010, U.S. Publication Nos. 2020/0131484 A1, 2020/0181587 A1, and 2024/0141427, each of which is incorporated by reference herein in its entirety. Exemplary polymerases include but not limited to Pol 812, Pol 1901, Pol 1558, Pol 963, or Pol A. The amino acid sequences of Pol 812, Pol 1901, Pol 1558 or Pol 963 DNA polymerases are described, for example, in U.S. Patent Publication Nos. 2020/0131484 A1 and 2020/0181587 A1. The amino acid sequence of Pol A is disclosed as SEQ ID NO:5 of U.S. Publication No. 2024/0141427. One buffer composition may comprise antioxidants such as ascorbic acid or sodium ascorbate, which can be used to protect the dye compounds from photo damage during detection. Additional buffer composition may comprise a reagent can may be used to cleave the 3′ blocking group and/or the cleavable linker. For example, a water-soluble phosphines or water-soluble transition metal catalysts formed from a transition metal and at least partially water-soluble ligands, such as a palladium complex. Various components of the kit may be provided in a concentrated form to be diluted prior to use. In such embodiments a suitable dilution buffer may also be included. Again, one or more of the components identified in a method set forth herein can be included in a kit of the present disclosure. In any embodiments of the nucleotide or labeled nucleotide described herein, the nucleotide contains a 3′ blocking group.

In some embodiments, the four different types of nucleotides are distinguishable using a single light source (e.g., a light source having a wavelength about 450 nm to about 460 nm, or a light source has a wavelength of about 520 nm to about 540 nm, for example about 523 nm). In other embodiments, the four different types of nucleotides are distinguishable using two light sources operating at two different wavelengths. In further embodiments, one light source has a wavelength of about 450 nm to about 460 nm, and the other light source has a wavelength of about 520 nm to about 540 nm.

Kits described herein may be used with systems for nucleic acid sequencing. In one aspect, the present disclosure provides for a system for nucleic acid sequencing, comprising a plurality of chambers, wherein one of the chamber contains a kit in accordance with the present disclosure.

### Two-Tag System

In some embodiments of the kit described herein, the incorporation mixture comprises a third type of unlabeled nucleotide. In some of these embodiments, the third type of unlabeled nucleotide comprises a mixture of the third type of unlabeled nucleotide comprising the first binding moiety and the third type of unlabeled nucleotide comprising the second binding moiety, and wherein both the first labeling reagent and the second labeling reagent are capable of specific binding to the third type of unlabeled nucleotide. In some further embodiments, each of the first reactive moiety and the second reactive moiety is covalently attached to the nucleobase of the third type of nucleotide via a cleavable linker.

In other embodiments, the incorporation mixture comprises a second type of labeled nucleotide and a third type of labeled nucleotide, in which the second type of nucleotide is labeled with a second detectable label that is spectrally distinguishable from the first detectable labels of the first labeling reagent. The third type of labeled nucleotide may be a mixture of the third type of nucleotide labeled with the first detectable label and the third type of nucleotide labeled with the second detectable label.

In other embodiments, the incorporation mixture comprises a second type of unlabeled nucleotide and a third type of labeled nucleotide, in which the second type of unlabeled nucleotide is labeled with a second labeling reagent post incorporation as described herein. The third type of labeled nucleotide may be a mixture of the third type of nucleotide labeled with the first detectable label and the third type of nucleotide labeled with the second detectable label.

### Three-Tag System

In some other embodiments of the kit described herein, the incorporation mixture comprises a third type of unlabeled nucleotide. The third type of unlabeled nucleotide comprises a third binding moiety, and the kit further comprises a third labeling reagent comprising a third labeled water-soluble nanoparticle that is capable of specific binding to the third reactive moiety as described herein. In some such embodiments, the third binding moiety is covalently attached to the nucleobase of the third type of unlabeled nucleotide via a cleavable linker.

In other embodiments, the incorporation mixture comprises a second type of labeled nucleotide and a third type of labeled nucleotide, in which the second type of nucleotide is labeled with a second detectable label that is spectrally distinguishable from the first detectable labels of the first labeling reagent. The third type of labeled nucleotide contains a third detectable label that is spectrally distinguishable from both the first and the second detectable labels.

In other embodiments, the incorporation mixture comprises a second type of unlabeled nucleotide and a third type of labeled nucleotide, in which the second type of unlabeled nucleotide is labeled with a second labeling reagent post incorporation as described herein. The third type of labeled nucleotide contains a third detectable label that is spectrally distinguishable from both the first and the second detectable labels.

### EXAMPLES

Additional embodiments are disclosed in further detail in the following examples, which are not in any way intended to limit the scope of the claims.

### Example 1. Nanogel Particle Preparation and Direct Dye Attachment

Nanoparticles in accordance with the present disclosure were synthesized by aqueous suspension and precipitation polymerization. The synthesis of the nanogel particles utilized suspension/precipitation free-radical polymerization. The nanogel particle comprises N-isopropylacrylamide (NiPAM), acrylic acid (AAc) and N,N′-methylenebisacrylamide (BisAM). Additionally, the synthesis of polymeric microgel particles used sodium dodecyl sulfate (SDS) as an anionic surfactant and ammonium persulfate (APS) as an initiator. The reaction was carried out in water as the dispersant at 70° C. for 4 hours. The average nanoparticle size was measured to be about 13 nm by dynamic light scattering measurements. The nanoparticles included carboxylic acid groups. The carboxylic acid groups of the nanoparticles were then activated with (4,6-Dimethoxy-1,3,5-triazin-2-yl)-4-methylmorpholinium chloride (DMTMM) and subsequently coupled with the amine-functionalized dyes. The nanoparticles were first labeled with a plurality of coumarin dye A1 having the structure

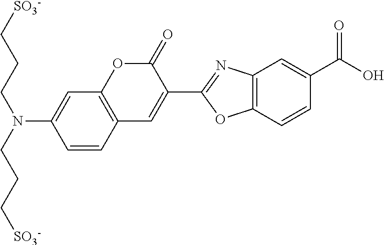

and then bound to streptavidin through the carboxyl group of the dye.

To attach coumarin dye A1 to a nanogel, 1 mL of nanogel was mixed with 1.2 mg of 4-(4,6-dimethoxy-1,3,5-triazin-2-yl)-4-methylmorpholinium chloride (DMTMM) and reacted at room temperature for 15 min. 100 μM of 0.626 mM coumarin dye A1-NH—C₂H₄—NH₂ was added to activated nanogel and reacted at room temperature for 1.5 hours. 600 μL of 2 mg/mL of streptavidin in 10 mM pH 7.4 potassium phosphate buffer was added to above dye-labeled nanogel and further reacted for 1 hour at room temperature. The unreacted free dye was removed using dialysis. Protein concentration and dye concentration were determined by nanodrop spectrophotometer.

### Example 2. Sequencing by Synthesis (SBS) Experiment on MiSeq®

In this experiment, SBS runs were conducted on Illumina MiSeq® instrument with blue and green LED operating at 460 nm and 532 nm. Images were taken simultaneously through collection channels which are in blue (472-520 nm) and green (540-640 nm). The standard incorporation mixture include: (1) a set of nucleotides comprising dark ffG, ffC-sPA-Dye A (blue dye), ffC-S07181, ffA-sPA-BL-NR550S0, ffA-sPA-BL-Dye A, ffT-sPA-AF550POPOS0 (a known green dye), each comprising a 3′-O—CH₂N₃ blocking group; (2) DNA polymerase; and (3) a glycine buffer.

In comparison, the incorporation mix used in the post incorporation nanogel labeling method included dark ffG, ffC-(LN3)₂-biotin, ffC-S07181, ffA-sPA-BL-NR550S0, ffA-sPA-BL-Dye A, ffT-sPA-AF550POPOS0, each comprising a 3′-O—CH₂N₃ blocking group. Post incorporation labeling mixture contains NP(dye A1)n(Strep)x in 10 mM pH 8 Tris buffer. The post incorporation labeling mixture was introduced to incorporated ffNs and allowed to bind at 60° C. by flushing through the flow cell without incubation.

Coumarin Dye A has strong fluorescence and great stability. This blue dye is disclosed in U.S. Publication No. 2020/0277670 A1 (incorporated by reference), having the structure moiety

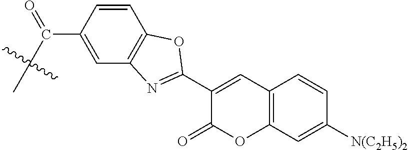

when conjugated with the ffA.

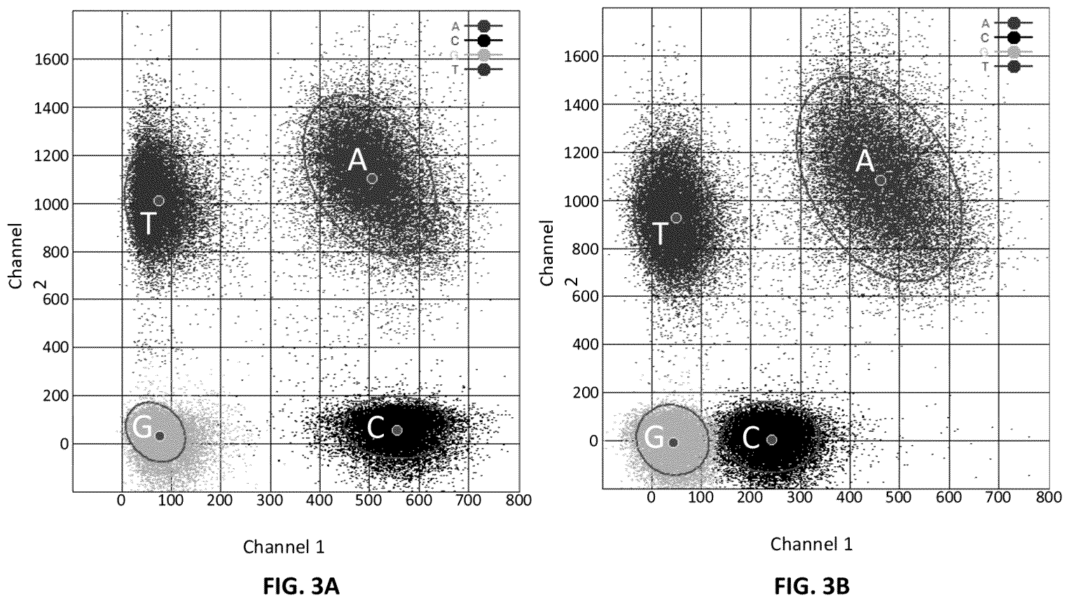

FIG. 3A is a scatter plot of a control run using standard SBS reagents. FIG. 3B is a scatter plots of the run using the nanoparticle and ffC. The results confirm that the nanoparticle successfully bound to the biotin of the ffC during SBS.

### Example 3. Preparation of Nanogel Particle with Indirect Dye Labeling

First, Dye B was reacted with NH₂—PEG₁₂-COOH (amide coupling) to form

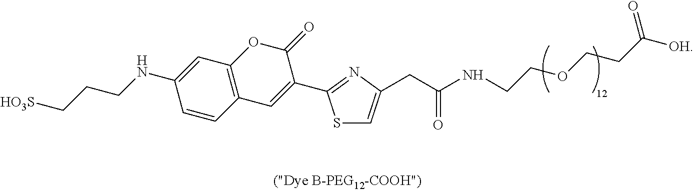

Then, 1 eq. of Dye B-PEG₁₂-COOH was co-evaporated with 1 ml of DMF. Dye B-PEG₁₂-COOH was dissolved in 250 μL of DMA and 10 eq of DIEPA and 2 eq TSTU was added to reacted under inert atmosphere for 30 min. Activation of the dye was checked using TLC with 20% acetonitrile in water. Once the activation was completed, 20 eq. of the activated dye was added to 1 eq. of streptavidin solution (pH=8, 10 mM potassium phosphate buffer) and reacted at room temperature for 1 hour. The unreacted dye was removed using a protein purification size exclusion column to obtain Strep(Dye B-PEG₁₂)_{3.7}.

Then, 1 mL of nanogel particle prepared according to Example 1 was mixed with 1.2 mg of 4-(4,6-Dimethoxy-1,3,5-triazin-2-yl)-4-methylmorpholinium chloride (DMTMM) and reacted at room temperature for 15 min. 300 μL of 10.8 μM of multi-dye labeled streptavidin (Strep(Dye B-PEG₁₂)_{3.7}) was added to activated nanogel and reacted at room temperature for 1 hours. The unreacted labeled streptavidin was removed using 100K centrifuge filters. Protein concentration and dye concentration was determined by nano drop spectrophotometer.

Coumarin Dye B (blue dye) is disclosed in U.S. Publication No. 2022/0033900 A1 (incorporated by reference), having the structure moiety

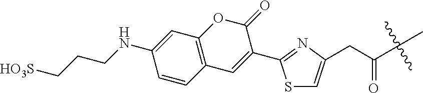 [SMILES: *C(=O)Cc1csc(-c2cc3ccc(NCCCS(=O)(=O)O)cc3oc2=O)n1]

when conjugated with the streptavidin through the PEG linker.

### Example 4. Post-Incorporation Labeling Sequencing by Synthesis Experiment on MiSeq®

Labeling reagents for use in a post-incorporation labeling SBS workflow was prepared accordingly to Example 3. Subsequently, three SBS runs were conducted on Illumina's MiSeq® instrument with blue and green LED operating at 460 nm and 532 nm. Images were taken simultaneously through collection channels which are in blue (472-520 nm) and green (540-640 nm). The standard incorporation mixture was used as a control, which include: (1) a set of nucleotides comprising dark ffG, ffC-sPA-Dye A, ffC-S07181, ffA-sPA-BL-NR550S0, ffA-sPA-BL-Dye A, ffT-sPA-AF550POPOS0, each comprising a 3′-O—CH₂N₃ blocking group; (2) DNA polymerase; and (3) a glycine buffer.

In comparison, a first post incorporation labeling SBS run used an incorporation mixture containing dark ffG, ffC-(LN3)₂-biotin, ffC-S07181, ffA-sPA-BL-NR550S, ffA-sPA-BL-Dye A, ffT-sPA-AF550POPOS0, each comprising a 3′-O—CH₂N₃ blocking group. Post incorporation labeling mixture contains Strep(Dye B-PEG₁₂)_{3.7} in 10 mM pH 8 Tris buffer. The post incorporation labeling mixture was introduced to incorporated ffNs and allowed to bind at 60° C. by flushing through the flow cell without incubation.

In a second post incorporation labeling SBS run, the incorporation mix used in the post incorporation nanogel labeling method included dark ffG, ffC-(LN3)₂-biotin, ffC-S07181, ffA-sPA-BL-NR550S0, ffA-sPA-BL-Dye A, ffT-sPA-AF550POPOS0, each comprising a 3′-O—CH₂N₃ blocking group. Post-incorporation labeling mixture contains nanogel particle [Strep(Dye B-PEG₁₂)_{3.7} in 10 mM pH 8 Tris buffer. The post incorporation labeling mixture was introduced to incorporated ffNs and allowed to bind at 60° C. by flushing through the flow cell without incubation.

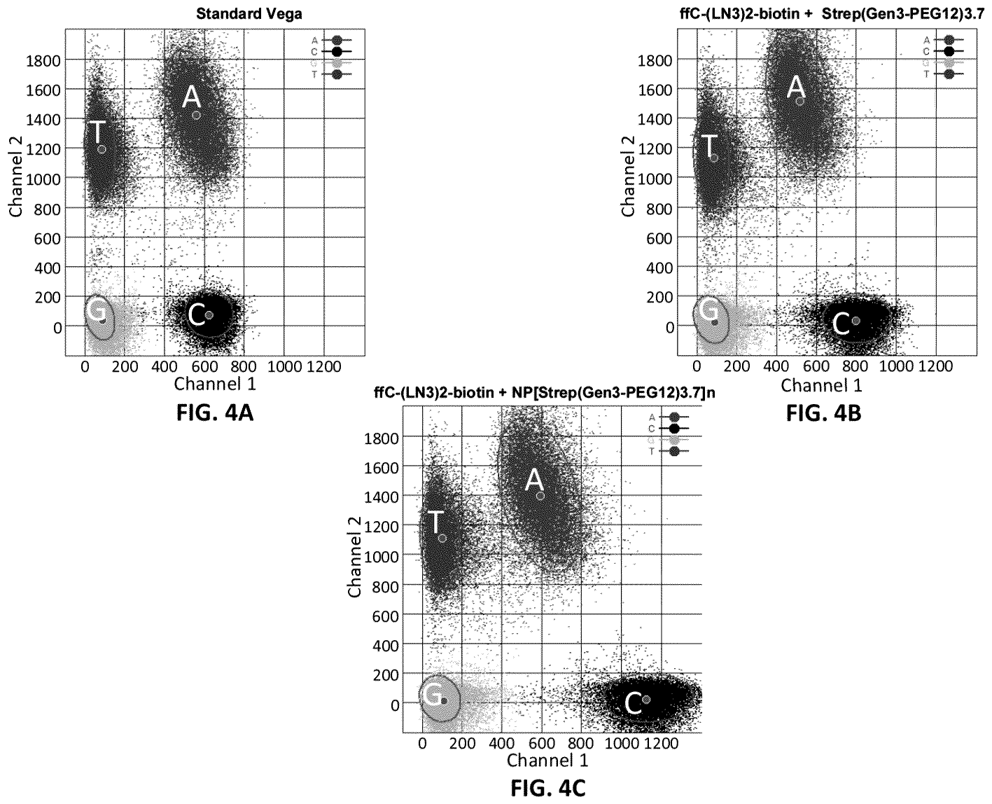

FIG. 4A is a scatter plot from the control SBS run. FIG. 4B is a scatterplot from the SBS run using Strep(Dye B-PEG₁₂)_{3.7} as the post incorporation labeling reagent, and FIG. 4C is a scatterplot from the SBS run with the nanogel particle with indirect dye labeling on streptavidin. The results confirmed that the labeled nanogel particle successfully bound to the biotin of the ffC during SBS.

### Example 5. Preparation of Nanogel Particle with Direct Dye Labeling

In this experiment, a nanogel particle labeling reagent was first prepared with reactive tetrazine moiety. In particular, the nanogel particle prepared according to Example 1 was directly labeled with a plurality of a known cyanine dye NR550C4 having the structure

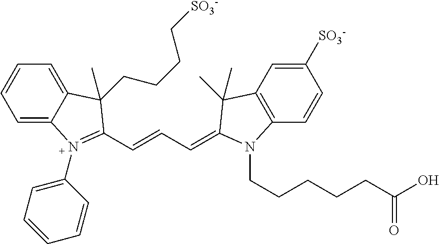

through amine coupling with NH₂—(CH₂)₂—NH₂. Subsequently, tetrazine functional groups were introduced to the nanogel particle by amide coupling with

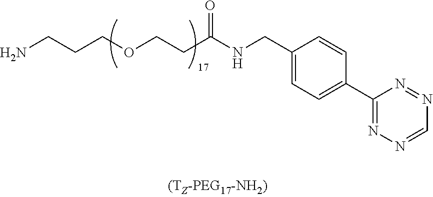

to form Tz-PEG₁₇-nanoparticle labeled with NR550C4.

### Example 6. Post-Incorporation Labeling Sequencing by Synthesis Experiment on MiSeq®

Labeling reagents for use in a post-incorporation labeling SBS workflow was prepared accordingly to Example 5. Subsequently, three SBS runs were conducted on Illumina MiSeq® instrument with blue and green LED operating at 460 nm and 532 nm. Images were taken simultaneously through collection channels which are in blue (472-520 nm) and green (540-640 nm).

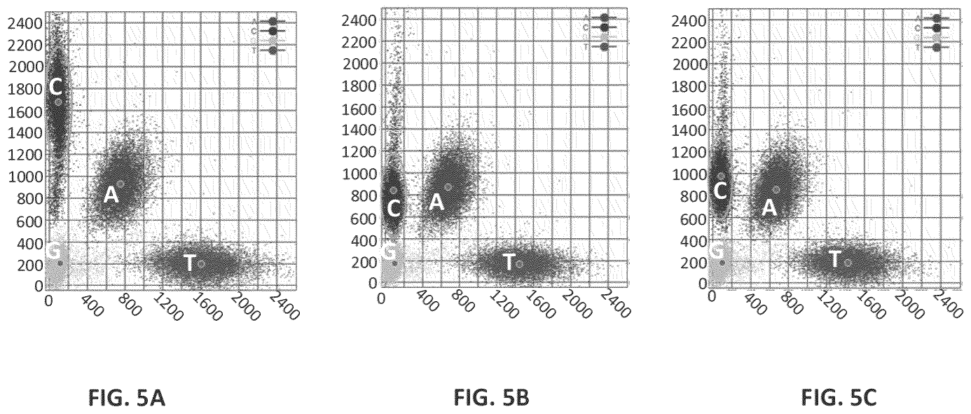

FIGS. 5A, 5B, and 5C are scatterplots the resulting SBS run. Each run used incorporation mix including ffG-dark, ffC-AOL-AOL-TCO, ffA-AOL-BL-NR550S0, ffA-AOL-BL-Dye A, and ffT-AOL-Dye A. Each of the nucleotides has a 3′-AOM blocking group. The preparation of ffC-AOL-AOL-TCO is described in U.S. Ser. No. 18/820,008, which is incorporated by reference in its entirety.

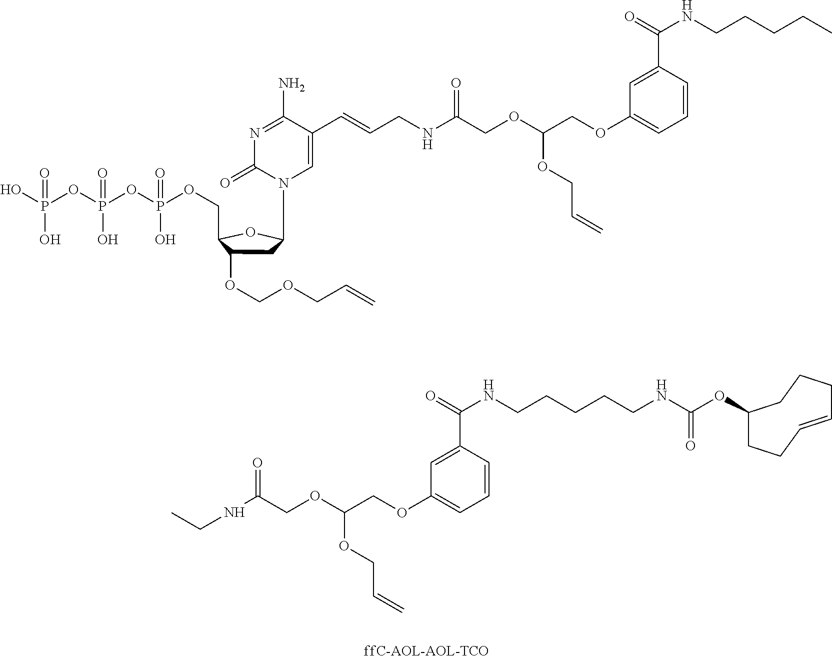

FIG. 5A is a scatterplot from the control SBS run, which included ffC-AOL-AOL-TCO and free label

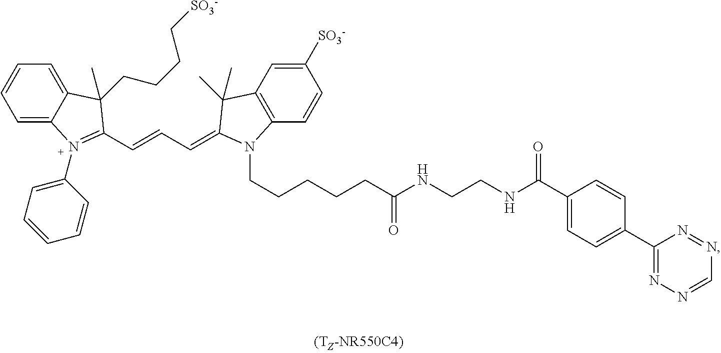

which was prepared by amide coupling of NR550C4 and Tz-COOH with NH₂—(CH₂)₂—NH₂. The concentration of the free label was 0.2 μM and exposure time was 2.5 s. FIGS. 5B and 5C are scatterplots from the SBS run including the labeled nanogel particle described in Example 5 (Tz-PEG₁₇-NP-NR550C4). The concentration of indirect carrier in both runs was 2.2 μM and exposure time was 50 s for the run of FIG. 5B and 100 s for the run of FIG. 5C. The results confirm that the indirect dye carriers can successfully bound to the biotin of the ffC during SBS.

1. A labeled water-soluble nanoparticle, comprising:
a polymer matrix, the polymer matrix comprises:
one or more first functional moieties configured to bind to an unlabeled nucleotide, and
a plurality of detectable labels, wherein the plurality of detectable labels are attached to the polymer matrix, optionally via covalent bonding to a plurality of second functional moieties of the polymer matrix or optionally via covalent bonding to the first functional moieties.

2. The labeled water-soluble nanoparticle of claim 1, wherein the polymer matrix comprises a hydrogel.

3. The labeled water-soluble nanoparticle of claim 1, wherein the first functional moiety is capable of forming covalent bonding with the unlabeled nucleotides via a biorthogonal reaction selected from the group consisting of a [3+2] dipolar cycloaddition, a Diels-Alder cycloaddition, a [4+1]cycloaddition, a phosphine ligation, or condensation with 2-acylphenyl boronic acid.

4. The labeled water-soluble nanoparticle of claim 3, wherein one of the first functional moiety and the unlabeled nucleotide comprises alkynyl, unsubstituted or substituted dibenzocyclooctyne (DBCO) moiety, unsubstituted or substituted bicyclo[6.1.0]nonyne (BCN) moiety, unsubstituted or substituted norbornene moiety, unsubstituted or substituted transcyclooctene (TCO) moiety, primary isonitrile, tertiary isonitrile, vinyl boronic acid, 2-acylphenyl boronic acid, phosphine methyl ester, phosphine alcohol, phosphine amine, or phosphine thiol, and the other one of the first functional moiety and the unlabeled nucleotide comprises azido, sydnone, imino sydnone, nitrone, phenyl tetrazine, pyrimidyl tetrazine, methyl tetrazine, pyridyl tetrazine, t-butyl tetrazine, triazine, cyclopropenone, cyclopropenium ion, DTO, chloro-oxime, or amino hydrazide.

5. The labeled water-soluble nanoparticle of claim 3, wherein the one or more first functional moieties is covalently attached to the polymer matrix via a spacer.

6. The labeled water-soluble nanoparticle of claim 5, wherein the spacer comprises polyethylene glycol (PEG) units.

7. The labeled water-soluble nanoparticle of claim 3, wherein the one or more first functional moieties comprises a 1,2,4,5-tetrazine moiety.

8. The labeled water-soluble nanoparticle of claim 7, wherein the one or more first functional moieties comprise a structure of

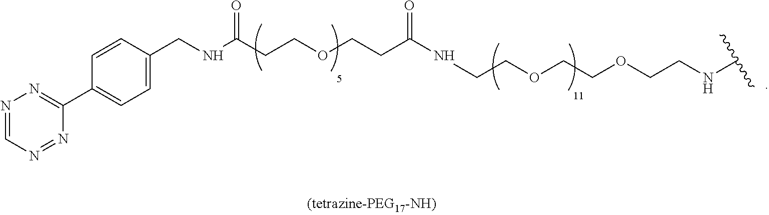

9. The labeled water-soluble nanoparticle of claim 1, wherein the plurality of detectable labels are attached to the polymer matrix via covalent bonding to the plurality of second functional moieties of the polymer matrix.

10. The labeled water-soluble nanoparticle of claim 9, wherein the plurality of second functional moieties comprise carboxyl groups.

11. The labeled water-soluble nanoparticle of claim 1, wherein the one or more first functional moieties are hapten-binding proteins capable of non-covalent binding with a hapten moiety of the unlabeled nucleotide.

12. The labeled water-soluble nanoparticle of claim 11, wherein the one or more first functional moieties comprise streptavidin or neutravidin.

13. The labeled water-soluble nanoparticle of claim 12, wherein the unlabeled nucleotide comprises a biotin moiety.

14. The labeled water-soluble nanoparticle of claim 11, wherein the plurality of detectable labels are attached to the polymer matrix via covalent bonding to the one or more first functional moieties.

15. (canceled)

16. (canceled)

17. The labeled water-soluble nanoparticle of claim 1, wherein the polymer matrix comprises a copolymer of polyacrylamide and polyacrylic acid.

18. The labeled water-soluble nanoparticle of claim 17, wherein the polymer matrix comprises a copolymerization product of N-isopropylacrylamide (NiPAM), acrylic acid (AAc) and N,N′-methylene bisacrylamide (BisAM).

19. (canceled)

20. The labeled water-soluble nanoparticle of claim 1, wherein the label comprises a structure of

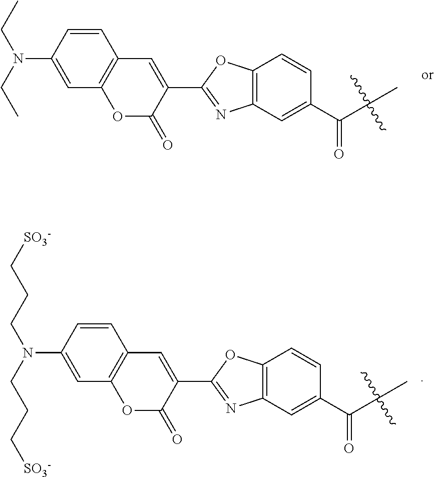

21. The labeled water-soluble nanoparticle of claim 1, wherein the diameter of the labeled nanoparticle is from about 1 nm to about 20 nm.

22. (canceled)

23. The labeled water-soluble nanoparticle of claim 1, wherein the unlabeled nucleotide is a terminal nucleotide incorporated into an oligonucleotide or polynucleotide.

24. (canceled)

25. (canceled)

26. A method of determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an incorporation mixture comprising DNA polymerase and one more of four different types of nucleotides under conditions suitable for DNA polymerase-mediated primer extension, and incorporating one type of nucleotides into the sequencing primers to produce extended copy polynucleotides; wherein
each of the four types of nucleotides comprises a 3′ blocking group; and
the incorporation mixture comprises a first type of unlabeled nucleotide having a first binding moiety covalently attached to the first type of unlabeled nucleotide;
(c) contacting the extended copy polynucleotides with a first labeling reagent comprising the labeled water-soluble nanoparticle of claim 1 that binds specifically with the first binding moiety of the first type of unlabeled nucleotides;
(d) imaging the solid support and performing one or more fluorescent measurements of the extended copy polynucleotides; and
(e) removing the 3′ blocking group of the incorporated nucleotides.

27.-44. (canceled)

45. A kit for sequencing application, comprising:
an incorporation mixture composition comprising one or more of four different types of nucleotides each comprising a 3′ blocking group, wherein a first type of unlabeled nucleotide having a first binding moiety covalently attached to the first type of unlabeled nucleotide; and
a first labeling reagent comprising a labeled water-soluble nanoparticle according to claim 1 that is capable of binding specifically with the first binding moiety of the first type of unlabeled nucleotides.

46.-57. (canceled)

58. A system for nucleic acid sequencing, comprising a plurality of chambers, wherein one of the chamber contains the kit of claim 45.
